(19) United States
(12) Patent Application Publication
(10) Pub. No.: US 2003/0133918 A1
(43) Pub. Date: Jul. 17, 2003

(54) Methods for ex vivo propagation of somatic stem cells

(76) Inventor: James L. Sherley, Boston, MA (US)

Correspondence Address: NIXON PEABODY LLP, ATTENTION: DAVID RESNICK, 101 FEDERAL STREET, BOSTON, MA, 02110, US

(21) Appl. No.: 10/192,110

(22) Filed: Jul. 10, 2002

Related U.S. Application Data
(60) Provisional application No. 60/304,384, filed on Jul. 10, 2001.

(51) Int. Cl. A61K 48/00; A61K 31/522; C12N 5/08; C12N 15/85; A61K 31/7076

(52) U.S. Cl. 424/93.21; 435/455; 435/366; 514/45; 514/263.34

(57) ABSTRACT

The present invention is directed to methods for readily propagating somatic tissue stem cells ex vivo. The methods comprise enhancing guanine nucleotide (GNP) biosynthesis, thereby expanding guanine nucleotide pools. This in turn conditionally suppresses asymmetric cell kinetics in the explanted tissue cells. The methods of the invention include pharmacological methods and genetic methods. The resulting cultured somatic stem cells can be used for a variety of applications including cell replacement therapies such as bone marrow transplants, gene therapies, tissue engineering, and in vitro organogenesis.

This invention was supported by Defense Advanced Research Projects Agency grant N0014-98-1-0760 and the government of the United States has certain rights thereto.

### FIELD OF THE INVENTION

The present application is directed to the ex vivo expansion of somatic tissue stem cells from a mammalian tissue, preferably somatic stem cells from human tissue.

### BACKGROUND OF THE INVENTION

Considerable attention has focused on stem cells and their uses in a range of therapies. For example, the gap between the need for replacement of damaged or diseased organs in patients, with otherwise significant life-expectancy, and the supply of donor organs is growing at an ever increasing rate (Gridelli and Remuzzi, 2000). Tissue bioengineering and in vitro organogenesis research have the potential to bridge this gap. The availability of stem cells for organs in demand would greatly accelerate progress in these efforts. Age-dependent changes in stem cell function are predicted to contribute to the aging of human tissues (Merok and Sherley, 2001; Rambhatla et al., 2001). Methods to isolate and study stem cells from individuals of different ages may reveal new tissue principles that can be applied to reducing morbidity associated with increasing age. Somatic stem cells are also ideal delivery vessels for gene therapy (Wilson, 1993; Brenner, 1996). In theory, after genetic engineering such stem cells would persist in a tissue while producing differentiated tissue constituent cells that would supply therapeutic gene expression. Depending on the tissue cellular architecture, the production of differentiated tissue cells could provide a tremendous amplification of gene dose. For example, in the small intestinal epithelium, a single stem cell compartment may give rise to as many as 4000 differentiated cells (Potten and Morris, 1988).

Somatic stem cells are derived from adult tissues, in contrast to other sources of stem cells such as cord stem cells and embryonic stem cells, which may originate from a variety of sources of embryonic tissue. Somatic stem cells are particularly attractive for a range of therapies in light of the ongoing controversies surrounding the use of embryonic stem cells.

Beyond their potential therapeutic applications, homogenous preparations of somatic stem cells would have another important benefit, the ability to study their molecular and biochemical properties. The existence of stem cells in somatic tissues is well established by functional tissue cell transplantation assays (Reisner et al., 1978). However, their individual identification has not been accomplished. Even though their numbers have been enriched by methods such as immuno-selection with specific antibodies, there are no known markers that uniquely identify stem cells in somatic tissues (Merok and Sherley, 2001). Thus, the ability to expand stem cell populations would allow the identification of stem cell-specific genes for the development of stem cell-specific molecular probes. Such stem cell markers could then be used to develop methods to identify stem cells in tissues and to isolate them directly from tissues. The simple ability to specifically enumerate stem cells in human tissues would add greatly to our understanding of tissue cell physiology. An understanding of the mechanisms which control stem cell number may suggest new therapeutic strategies for cancer prevention and treatment, and for reducing morbidity associated with aging.

Accordingly, methods to isolate and expand stem cells from somatic tissue, particularly without significant differentiation, are highly desirable. The availability of a method for producing somatic stem cells from adult tissues would greatly contribute to cell replacement therapies such as bone marrow transplants, gene therapies, tissue engineering, and in vitro organogenesis. Production of autologous stem cells to replace injured tissue would also reduce the need for immune suppression interventions. Considerable difficulty in achieving this objective has been encountered, thus far.

Cell growth is a carefully regulated process that responds to the specific needs of the body in different tissues and at different stages of development. In a young animal, cell multiplication exceeds cell loss and the animal increases in size; in an adult, the processes of cell division and cell loss are balanced to maintain a steady state. For some adult cell types, renewal is rapid: intestinal cells and certain white blood cells have a half-life of a few days before they die and are replaced. In contrast, the half-life of human red blood cells is approximately 100 days; healthy liver cells rarely die, and in adults, there is a slow loss of brain cells with little or no replacement.

Somatic stem cells possess the ability to renew adult tissues (Fuchs and Segre, 2000). The predominant way somatic stem cells divide is by asymmetric cell kinetics (see FIG. 1). During asymmetric kinetics, one daughter cell divides with the same kinetics as its stem cell parent, while the second daughter gives rise to a differentiating non-dividing cell lineage. The second daughter may differentiate immediately; or, depending on the tissue, it may undergo a finite number of successive symmetric divisions to give rise to a larger pool of differentiating cells.

Attempts at somatic stem cell isolation have been described, for example, in studies to enrich for hematopoietic stem cells (HSCs; Phillips et al., 2000). However, although high degrees of enrichment have been reported, so far HSCs (and other somatic stem cells) have neither been identified nor purified to homogeneity. A major obstacle to these two challenges is the inability to expand HSCs in culture. If it were possible to propagate HSCs (or any somatic stem cell for that matter) in culture, then the use of standard molecular and cell biology techniques should be sufficient to result in their identification, purification, and characterization.

Attempts at propagating somatic stem cells have encountered a number of significant difficulties. The asymmetric cell kinetics which are a defining characteristic of somatic stem cells are also a major obstacle to their expansion in vitro (FIG. 2) (Merok and Sherley, 2001; Rambhatla et al., 2001). In culture, continued asymmetric cell kinetics results in dilution and loss of an initial relatively fixed number of stem cells by the accumulation of much greater numbers of their terminally differentiating progeny. If a sample includes both exponentially growing cells as well as somatic stem cells, the growth of the exponentially growing cells will rapidly overwhelm the somatic stem cells, leading to their dilution.

Even in instances where it is possible to select for relatively purer populations such as hematopoietic stem cells (for example by cell sorting), these populations do not expand when cultured.

Thus, despite the need for methods to isolate somatic cells from an individual and expand them ex vivo, it has not been possible to readily do so.

### SUMMARY OF THE INVENTION

We have now discovered methods for readily propagating somatic tissue stem cells ex vivo. The methods comprise enhancing guanine nucleotide (GNP) biosynthesis, thereby expanding guanine nucleotide pools. This in turn conditionally suppresses asymmetric cell kinetics in the explanted tissue cells. The methods of the invention include pharmacological methods and genetic methods. One preferred method of enhancing guanine nucleotide biosynthesis is to bypass or override normal inosine-5′-monophosphate dehydrogenase (IMPDH) regulation. IMPDH catalyzes the conversion of inosine-5′ monophosphate (IMP) to xanthosine monophosphate (XMP) for guanine nucleotide biosynthesis. This step can be bypassed or overridden by providing a guanine nucleotide precursor (rGNPr) such as xanthosine or hypoxanthine, respectively. The next metabolite in the GNP pathway is guanine monophosphate (GMP), which in turn is metabolized to the cellular guanine nucleotides. The resulting cultured somatic stem cells can be used for a variety of applications including cell replacement therapies such as bone marrow transplants, gene therapies, tissue engineering, and in vitro organogenesis.

In one preferred embodiment of the invention, somatic stem cells are removed and cultivated in the presence of compounds such as guanine nucleotide precursors (rGNPrs), which lead to increased guanine nucleotide pools. Preferably, the rGNPr is xanthosine or hypoxanthine. Even more preferably, the rGNPr is xanthosine.

In another preferred embodiment of the invention, the somatic stem cells are propagated in a primitive undifferentiated state but retain the ability to be induced to produce differentiating progeny cells.

Another preferred embodiment provides for deriving clonal lines of somatic tissue stem cells by limiting dilution plating or single cell sorting in the presence of compounds which enhance guanine nucleotide biosynthesis, thereby suppressing asymmetric cell kinetics.

In another embodiment of the invention, genes that lead to constitutive upregulation of guanine ribonucleotides (rGNPs) are introduced into the somatic stem cells. Preferred genes are those that encode inosine-5′monophosphate dehydrogenase (IMPDH) or xanthine phosphoribosyltransferase (XPRT). More preferably, XPRT.

Another embodiment of the invention provides methods for administering stem cells to a patient in need thereof, comprising the steps of (1) isolating somatic stem cells from an individual; (2) expanding the somatic stem cells in culture using pharmacological or genetic methods to enhance guanine nucleotide biosynthesis to expand guanine nucleotide pools and suppress asymmetric cell kinetics; and thereafter, (3) administering the expanded stem cells to said individual in need thereof.

Further embodiments of the invention provide for additional manipulations, including genetic manipulation of the somatic tissue stem cells prior to administration to the individual.

Another preferred embodiment provides for the use of expanded somatic stem cells to identify molecular probes specific for somatic stem cells in tissues or tissue cell preparations.

Another preferred embodiment of the invention provides transgenic non-human animals into whose genome is stably integrated an exogenous DNA sequence comprising a ubiquitously-expressed promoter operably linked to a DNA sequence encoding a protein that leads to constitutive upregulation of guanine nucleotides, including the gene encoding inosine-5′-monophosphate dehydrogenase (IMPDH) or xanthine phophoribosyl transferase (XPRT). Preferably, the transgene is XPRT driven by a ubiquitously expressed promoter. Preferably, the transgenic animal is a mouse.

### BRIEF DESCRIPTION OF THE DRAWINGS

FIGS. 1A-B depict the in vivo asymmetric kinetics of somatic stem cells. In vivo, somatic stem cells (SSC, bold-lined circles) can exhibit one of three division programs: 1) Highly restricted exponential kinetics that produce two similar somatic stem cells (brackets); 2) Dormancy (stippled circle); and 3) Asymmetric kinetics, the most populated somatic stem cell kinetics state in most tissues. Asymmetric somatic stem cells underlie turnover units (TU; Hererro-Jimenez et al., 1998). Turnover units are comprised of three cell types: an asymmetric somatic stem cell, transit cells (thin-lined open circle), and mature, differentiated, non-dividing terminal cells (closed circle). Asymmetric somatic stem cells divide to produce another asymmetric somatic stem cell and a transit cell. Depending on the type of tissue, the transit cell may undergo no further division, or a finite number of successive divisions may occur. However, all transit lineage cells mature into differentiated, non-dividing terminal cells. The model cells with conditional asymmetric cell kinetics can be induced to switch from exponential kinetics (left compartment) to two types of asymmetric kinetics programs (right compartment) that have the key features of asymmetric somatic stem cell kinetics in vivo.

FIG. 2 depicts a cell kinetics barrier to the expansion of somatic stem cells in vitro. Of explanted tissue cells, somatic stem cells (bold-lined, open circles) have the capacity for long-term division ex vivo. However, if they retain even a rudimentary form of their in vivo asymmetric cell kinetics program, in vitro, their numbers will not increase. Instead, they will be diluted by the continuous accumulation of cells in terminal arrest lineages (closed circles). Continuous passage of cultures will result in “senescence” as a kinetics endpoint. In order to establish an immortal cell line, mutations must occur that either interfere with the maturation of terminal cells (immature terminal cells, thin-lined open circles) or that convert stem cells to symmetric exponential kinetics, in which only stem cells are produced. If asymmetric stem cell kinetics were suppressed, this model predicts that stem cells could be expanded in culture with fewer growth-activating mutations, like p53 mutations.

FIGS. 3A-F show DPA of rat liver epithelial cell lines with asymmetric cell kinetics. Paired newly-divided daughter cells (as described in examples) of: exponentially dividing Xs-F1 cells grown in the absence of Xs (FIGS. 3A, D); exponentially dividing Xs-D8 cells grown in the presence of xanthosine (FIGS. 3B, E); and asymmetrically dividing Xs-D8 cells grown in the absence of Xs (FIGS. 3C, F). FIGS. 3A-C, DAPI fluorescence to detect daughter cell nuclei; FIGS. 4D-F, in situ immunofluorescence analysis to detect BrdU(+) daughters.

FIGS. 4A-D show albumin induction in Xs-D cell lines. Lines Xs-F1 (FIGS. 4A and 4C) and Xs-D8 (FIGS. 4B and 4D) were grown to confluency in Xs-free medium (Active; 10% Serum) and examined immediately (FIGS. 4A and 4B)for albumin-specific immunohistochemical staining or after 1 week of quiescence in Xs-free medium reduced to 1% serum (Arrested) (FIGS. 4C and 4D). In Xs-free medium Xs-D8 cells divide with asymmetric cell kinetics, whereas Xs-F1 cell kinetics are exponential. The brown color in FIG. 4D indicates positive staining for albumin expression.

FIGS. 5A-C show scanning electron micrographs of Xs-D8 spheroids in suspension culture. FIGS. 5A and 5B show images of independent spheroids at low magnification to show morphological differentiation. FIG. 5C shows a higher magnification of the middle image to show microvilli-like surface projections.

FIG. 6 shows effect of xanthosine (Xs) on non-adherent cell production in long-term bone marrow culture. The non-adherent cell production rate (CPR) is defined as the total number of non-adherent cells produced in a single long-term culture normalized to days of culture (mean=95 days; range=66 to 132) and input cell number (range=5×10⁶ to 20×10⁶ cells).

FIGS. 7A-C show bone marrow cell colony types detected in soft agar culture experiments. In long-term cultures (FIG. 7A), cells that produce type I colonies are found primarily in the adherent cell fraction, whereas cells that give rise to type II (FIG. 7B) and III (FIG. 7C) colonies predominate in the non-adherent fraction. Only type I colonies increase significantly in number in Xs-containing medium.

FIG. 8 shows effects of Xs and Hx on bone marrow colony formation in soft agar. The following numbers of freshly harvested murine bone marrow cells were plated in soft agar in replicates of 6 in 6-well plates: 1×10⁵, 5×10⁵, or 1×10⁶. After 1-3 weeks, type I, II, and III colonies (see FIG. 2 for examples) were enumerated by phase microscopy. Bar heights indicate the mean number of colonies detected per 10⁵ input bone marrow cells for 2-4 independent experiments.

FIG. 9 shows effect of Xs on serial transfer for LT-CFC-derived type I colonies in soft agar. Type I colonies were isolated from soft agar with either control medium or medium containing Xs (data for 1 mM and 3 mM Xs are combined). Colonies were dispersed and then plated in a single 6-well in soft agar developed with culture medium containing the respective concentration of Xs. After 1-3 weeks, the number and type of secondary colonies were scored by phase microscopy. Bars indicate the mean percent of positive wells for each secondary colony type. Data for the control condition are the means of two independent experiments; data for Xs condition are the means of four independent experiments.

FIGS. 10A-F show constitutive IMPDH expression prevents p53-dependent asymmetric cell kinetics. Brightly fluorescent Hoechst dye-stained nuclei were detected as an indicator of asymmetric cell kinetics in cells expressing wild-type p53 protein. Zn-dependent fibroblast lines were grown for 72 hours under either control conditions for exponential kinetics (0 μM ZnCl₂; FIGS. 10A, 10C, 10E) or p53-inducing conditions for asymmetric kinetics (75 μM ZnCl₂; 10B, 10D, 10F). During the last 48 hours (equivalent to approximately two cell cycle periods), BrdU was added. At the end of the labeling period, cells were fixed, stained with Hoechst 33258, and examined for UV-excited nuclear fluorescence. Indicative of their exponential kinetics, p53-null cells (Con-3; Liu et al. 1998ab) exhibit uniform dimly fluorescent nuclei under non-inducing (FIG. 10A) or p53-inducing conditions (FIG. 10B). In contrast, p53-inducible vector-transfectant cells (FIGS. 10C and 10D; tC-4; Liu et al. 1998b) exhibit conditional asymmetric cell kinetics. In FIG. 10D, the reduced number of cells and the presence of both bright and dim nuclei are indicative of asymmetric cell kinetics. p53-inducible impd-transfectant cells (FIGS. 10E and 10F; tI-3; Liu et al. 1998b) exhibit a nuclear fluorescence pattern like that of p53-null cells (compare FIGS. 10A and 10B), indicating complete abrogation of asymmetric cell kinetics. Magnification=300×.

FIGS. 11A-B show in silico analysis of the effect of p53 genotype on the association between type II IMPDH mRNA expression and human cancer cell proliferation kinetics Simple linear regressions were performed for type II IMPDH mRNA expression versus cell doubling time (O'Connor et al. 1997). The analyses were performed with gene expression micro-array data for the NCI60 cancer cell line panel stratified for either wild-type p53 protein expression (FIG. 11A) or homozygous mutant p53 protein expression (FIG. 11B).

### DETAILED DESCRIPTION OF THE INVENTION

We have now discovered methods for propagating somatic stem cells ex vivo by enhancing guanine nucleotide biosynthesis, thereby expanding guanine nucleotide pools, and conditionally suppressing asymmetric cell kinetics in the explanted stem cells. The methods of the invention include pharmacological methods and genetic methods. Somatic stem cells can be used for a variety of applications, including cell replacement therapies such as bone marrow transplants, gene therapies, tissue engineering, and in vitro organogenesis.

As used herein, somatic stem cells derived from adult tissues are sometimes referred to as somatic tissue stem cells or somatic stem cells or simply as stem cells.

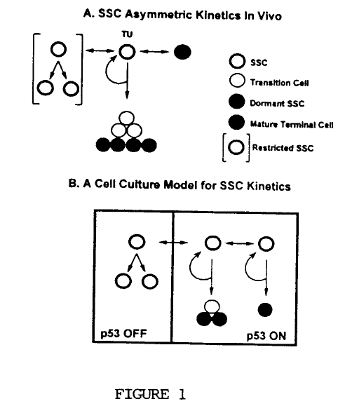

Adult somatic stem cells predominantly divide by asymmetric cell kinetics (see FIG. 1). While somatic stem cells also undergo limited symmetric divisions (that produce two identical stem cells) in developing adult tissues, such symmetric kinetics are restricted to periods of tissue expansion and tissue repair. Inappropriate symmetric somatic stem cell divisions evoke mechanisms leading to apoptosis of duplicitous stem cells (Potten and Grant, 1998). Some stem cells may also lie dormant for long periods before initiating division in response to specific developmental cues, as in reproductive tissues like the breast. However, the predominant cell kinetics state of somatic stem cells is asymmetric (Cairns, 1975; Poldosky, 1993; Loeffler and Potten, 1997).

During asymmetric cell kinetics, one daughter cell divides with the same kinetics as its stem cell parent, while the second daughter gives rise to a differentiating non-dividing cell lineage. The second daughter may differentiate immediately; or depending on the tissue, it may undergo a finite number of successive symmetric divisions to give rise to a larger pool of differentiating cells. The second daughter and its dividing progeny are called transit cells (Loeffler and Potten, 1997). Transit cell divisions ultimately result in mature, differentiated, terminally arrested cells. In tissues with high rates of cell turnover, the endpoint for differentiated terminal cells is programmed cell death by apoptosis.

Asymmetric cell kinetics evolved in vertebrates as a mechanism to insure tissue cell renewal while maintaining a limited set of stem cells and constant adult body mass. Mutations that disrupt asymmetric cell kinetics are an absolute requirement for the formation of a clinically significant tumor mass (Cairns, 1975). In many ways, asymmetric cell kinetics provide a critical protective mechanism against the emergence of neoplastic growths that are life threatening.

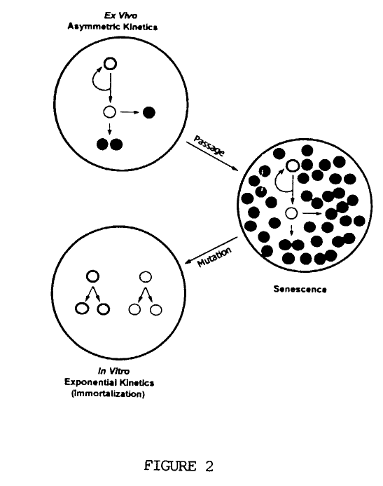

In culture, continued asymmetric cell kinetics of explanted cells are a major obstacle to their expansion in vitro (FIG. 2). Ongoing asymmetric kinetics results in dilution and loss of an initial relatively fixed number of stem cells by the accumulation of much greater numbers of their terminally differentiating progeny. If a sample includes both exponentially growing cells as well as somatic stem cells, the growth of the exponentially growing cells will rapidly overwhelm the somatic stem cells, leading to their dilution.

One regulator of asymmetric cell kinetics is the p53 tumor suppressor protein. Several stable cultured murine cell lines have been derived that exhibit asymmetric cell kinetics in response to controlled expression of the wild-type murine p53 (FIG. 1B). (Sherley, 1991; Sherley et al, 1995 A-B; Liu et al., 1998 A-B; Rambhatla et al., 2001).

The p53 model cell lines have been used to define cellular mechanisms that regulate asymmetric cell kinetics. In addition to p53, the rate-limiting enzyme of guanine nucleotide biosynthesis, inosine-5′-monophosphate dehydrogenase (IMPDH) is an important determinant of asymmetric cell kinetics. IMPDH catalyzes the conversion of IMP to xanthosine monophosphate (XMP) for guanine nucleotide biosynthesis. This enzymatic reaction is rate-determining for the formation of the next metabolite in the pathway, GMP, from which all other cellular guanine nucleotides are derived.

Accordingly, high levels of GNPs promote exponential kinetics, whereas low levels of GNPs promote asymmetric cell kinetics. The present invention provides methods for expanding somatic stem cells ex vivo by enhancing guanine nucleotide biosynthesis, thereby expanding cellular pools of GNPs and conditionally suppressing asymmetric cell kinetics.

Mechanisms which function downstream of the GNPs to regulate cell kinetics (i.e. asymmetric v. exponential) can also be used to conditionally suppress asymmetric cell kinetics. These mechanisms include both genetic and/or pharmacological approaches, analogous to those described in detail herein. For example, one can enhance expression of a protein downstream of the GNP biosynthesis pathway, if that protein inhibits asymmetric cell kinetics. Alternatively, one can downregulate expression of a protein downstream of the GNP pathway if it promotes asymmetric cell kinetics.

Pharmacological Methods for Stem Cell Expansion

In the pharmacological method of the present invention, somatic tissue stem cells are cultivated in the presence of compounds which enhance guanine nucleotide biosynthesis. This expands guanine nucleotide pools, which in turn suppress the undesired asymmetric cell kinetics thereby permitting expansion of stem cells. Preferably, the compounds are guanine nucleotide precursors (rGNPrs). Even more preferably, the rGNPr is xanthosine (Xs) or hypoxanthine (Hx). More preferably, the rGNPr is xanthosine. These compounds can be used in effective amounts depending upon the culturing condition. Xanthosine can be used from 1-10,000 μM. Hypothanine can be used from 1-5000 μM. More specifically, xanthosine and hypoxanthine can be used from 50-400 μM, for example to expand somatic tissue stem cells from liver epitheilia, or in studies of model cell lines. Xanthosine and hypoxanthine can be used from 1-3 mM to expand hematopoetic stem cells. Further optimization of effective dosages can be determined empirically based on the specific tissue and cell type.

Genetic Methods for Stem Cell Expansion

In another embodiment of the invention, genes that lead to constitutive upregulation of guanine ribonucleotides (rGNPs) are introduced into the somatic stem cells explants. Preferred genes are those that encode inosine-5′monophosphate dehydrogenase (IMPDH) or xanthine phosphoribosyltransferase (XPRT), or other genes which have the same biochemical effect. More preferably, the gene is XPRT. While there are currently no known mammalian forms of XPRT, and its substrate xanthine is present in very low levels in mammalian cells, the activity of the transgenic XPRT can be regulated by supplying xanthine exogenously. As explained below, it is preferred that the genes are operably linked to an inducible promoter.

As used herein, the introduction of DNA into a host cell is referred to as transduction, sometimes also known as transfection or infection. Stem cells can be transduced ex vivo at high efficiency.

As used herein, the terms “transgene”, “heterologous gene”, “exogenous genetic material”, “exogenous gene” and “nucleotide sequence encoding the gene” are used interchangeably and meant to refer to genomic DNA, cDNA, synthetic DNA and RNA, mRNA and antisense DNA and RNA which is introduced into the stem cell. The exogenous genetic material may be heterologous or an additional copy or copies of genetic material normally found in the individual or animal. When cells are to be used as a component of a pharmaceutical composition in a method for treating human diseases, conditions or disorders, the exogenous genetic material that is used to transform the cells may also encode proteins selected as therapeutics used to treat the individual and/or to make the cells more amenable to transplantation.

An expression cassette can be created for expression of the gene that leads to constitutive upregulation of guanine ribonucleotides. Such an expression cassette can include regulatory elements such as a promoter, an initiation codon, a stop codon, and a polyadenylation signal. It is necessary that these elements be operable in the stem cells or in cells that arise from the stem cells after infusion into an individual. Moreover, it is necessary that these elements be operably linked to the nucleotide sequence that encodes the protein such that the nucleotide sequence can be expressed in the stem cells and thus the protein can be produced. Initiation codons and stop codons are generally considered to be part of a nucleotide sequence that encodes the protein.

A variety of promoters can be used for expression of the transgene. Promoters that can be used to express the gene are well known in the art. Promoters include cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter and the herpes simplex tk virus promoter. For example, one can use a tissue specific promoter, i.e. a promoter that functions in some tissues but not in others. Such promoters include EF2 responsive promoters, etc. Regulatable promoters are preferred. Such systems include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone or rapamycin [see Miller and Vvhelan, supra at FIG. 2]. Inducible systems are available from Invitrogen, Clontech and Ariad. Systems using a repressor with the operon are preferred. Regulation of transgene expression in target cells represents a critical aspect of gene therapy. For example, the lac repressor from Escherichia coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP16) to create a tetR-mammalian cell transcription activator fusion protein, tTa (tetR-VP16), with the tetO-bearing minimal promoter derived from the human cytomegalovirus (hCMV) major immediate-early promoter to create a tetR-tet operator system to control gene expression in mammalian cells. Recently Yao and colleagues [F. Yao et al., Human Gene Therapy, supra] demonstrated that the tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter. One particular advantage of this tetracycline inducible switch is that it does not require the use of a tetracycline repressor-mammalian cells transactivator or repressor fusion protein, which in some instances can be toxic to cells [M. Gossen et al., Natl. Acad Sci. USA, 89:5547-5551 (1992); P. Shockett et al., Proc. Natl. Acad. Sci. USA, 92:6522-6526 (1995)], to achieve its regulatable effects.

The effectiveness of some inducible promoters increases over time. In such cases one can enhance the effectiveness of such systems by inserting multiple repressors in tandem, e.g. TetR linked to a TetR by an IRES. Alternatively, one can wait at least 3 days before screening for the desired function. While some silencing may occur, it is minimized given the large number of cells being used, preferably at least 1×10⁴, more preferably at least 1×10⁵, still more preferably at least 1×10⁶, and even more preferably at least 1×10⁷, the effect of silencing is minimal. One can enhance expression of desired proteins by known means to enhance the effectiveness of this system. For example, using the Woodchuck Hepatitis Virus Posttranscriptional Regulatory Element (WPRE). See Loeb, V. E., et al., Human Gene Therapy 10:2295-2305 (1999); Zufferey, R., et al., J. of Virol. 73:2886-2892 (1999); Donello, J. E., et al., J. of Virol. 72:5085-5092 (1998).

Examples of polyadenylation signals useful to practice the present invention include but are not limited to human collagen I polyadenylation signal, human collagen II polyadenylation signal, and SV40 polyadenylation signal.

In order to maximize protein production, codons may be selected which are most efficiently translated in the cell. The skilled artisan can prepare such sequences using known techniques based upon the present disclosure.

The exogenous genetic material that includes the transgene operably linked to the regulatory elements may remain present in the cell as a functioning cytoplasmic molecule, a functioning episomal molecule or it may integrate into the cell's chromosomal DNA. Exogenous genetic material may be introduced into cells where it remains as separate genetic material in the form of a plasmid. Alternatively, linear DNA, which can integrate into the chromosome, may be introduced into the cell. When introducing DNA into the cell, reagents, which promote DNA integration into chromosomes, may be added. DNA sequences, which are useful to promote integration, may also be included in the DNA molecule. Alternatively, RNA may be introduced into the cell.

Selectable markers can be used to monitor uptake of the desired gene. These marker genes can be under the control of any promoter or an inducible promoter. These are well known in the art and include genes that change the sensitivity of a cell to a stimulus such as a nutrient, an antibiotic, etc. Genes include those for neo, puro, tk, multiple drug resistance (MDR), etc. Other genes express proteins that can readily be screened for such as green fluorescent protein (GFP), blue fluorescent protein (BFP), luciferase, LacZ, nerve growth factor receptor (NGFR), etc.

For example, one can set up systems to screen stem cells automatically for the marker. In this way one can rapidly select transduced stem cells from non-transformed cells. For example, the resultant particles can be contacted with about one million cells. Even at transduction rates of 10-15% one will obtain 100-150,000 cells. An automatic sorter that screens and selects cells displaying the marker, e.g. GFP, can be used in the present method.

When the transgene is XPRT, cells expressing XPRT will be resistant to cytotoxic IMPDH inhibitors such as mycophenolic acid in the presence of xanthine. Thus, transduced stem cells can be selected from non-transformed cells by culturing transfectants in the presence of an IMPDH inhibitor (such as mycophenolic acid) and xanthine.

Vectors include chemical conjugates, plasmids, phage, etc. The vectors can be chromosomal, non-chromosomal or synthetic. Commercial expression vectors are well known in the art, for example pcDNA 3.1, pcDNA4 HisMax, pACH, pMT4, PND, etc. Preferred vectors include viral vectors, fusion proteins and chemical conjugates. Retroviral vectors include Moloney murine leukemia viruses and pseudotyped lentiviral vectors such as FIV or HIV cores with a heterologous envelope. Other vectors include pox vectors such as orthopox or avipox vectors, herpesvirus vectors such as a herpes simplex I virus (HSV) vector (Geller, A. I. et al., (1995), J. Neurochem, 64: 487; Lim, F., et al., (1995) in DNA Cloning: Mammalian Systems, D. Glover, Ed., Oxford Univ. Press, Oxford England; Geller, A. I. et al. (1993), Proc Natl. Acad. Sci.: U.S.A. 90:7603; Geller, A. I., et al., (1990) Proc Natl. Acad. Sci USA 87:1149), adenovirus vectors (LeGal LaSalle et al. (1993), Science, 259:988; Davidson, et al. (1993) Nat. Genet 3: 219; Yang, et al., (1995) J. Virol. 69: 2004) and adeno-associated virus vectors (Kaplitt, M. G., et al. (1994) Nat. Genet. 8: 148).

The introduction of the gene into the stem cell can be by standard techniques, e.g. infection, transfection, transduction or transformation. Examples of modes of gene transfer include e.g., naked DNA, CaPO₄ precipitation, DEAE dextran, electroporation, protoplast fusion, lipofection, cell microinjection, and viral vectors, adjuvant-assisted DNA, gene gun, catheters, etc.

The vectors are used to transduce the stem cells ex vivo. One can rapidly select the transduced cells by screening for the marker. Thereafter, one can take the transduced cells and grow them under the appropriate conditions or insert those cells into a host animal.

Somatic Tissue Stem Cells

Somatic tissue stem cells of the present invention include any stem cells isolated from adult tissue. Somatic stem cells include but are not limited to bone marrow derived stem cells, adipose derived stem cells, mesenchymal stem cells, neural stem cells, liver stem cells, and pancreatic stem cells. Bone marrow derived stem cells refers to all stem cells derived from bone marrow; these include but are not limited to mesenchymal stem cells, bone marrow stromal cells, and hematopoietic stem cells. Bone marrow stem cells are also known as mesenchymal stem cells or bone marrow stromal stem cells, or simply stromal cells or stem cells.

The stem cells act as precursor cells, which produce daughter cells that mature into differentiated cells. The stem cells can be isolated from the individual in need of stem cell therapy or from another individual. Preferably, the individual is a matched individual to insure that rejection problems do not occur. Therapies to avoid rejection of foreign cells are known in the art. Furthermore, somatic stem cells may be immune-privileged, so the graft versus host disease after allogenic transplant may be minimal or non-existent (Weissman, 2000). Endogenous or stem cells from a matched donor may be administered by any known means, preferably intravenous injection, or injection directly into the appropriate tissue.

In some embodiments, somatic tissue stem cells can be isolated from fresh bone marrow or adipose tissue by fractionation using fluorescence activated call sorting (FACS) with unique cell surface antigens to isolate specific subtypes of stem cells (such as bone marrow or adipose derived stem cells) for injection into recipients following expansion in vitro, as described above.

As stated above, stem cells may also be derived from the individual to be treated or a matched donor. Those having ordinary skill in the art can readily identify matched donors using standard techniques and criteria.

Two preferred embodiments provide bone marrow or adipose tissue derived stem cells, which may be obtained by removing bone marrow cells or fat cells, from a donor, either self or matched, and placing the cells in a sterile container with a plastic surface or other appropriate surface that the cells come into contact with. The stromal cells will adhere to the plastic surface within 30 minutes to about 6 hours. After at least 30 minutes, preferably about four hours, the non-adhered cells may be removed and discarded. The adhered cells are stem cells, which are initially non-dividing. After about 2-4 days however the cells begin to proliferate.

Cells can be obtained from donor tissue by dissociation of individual cells from the connecting extracellular matrix of the tissue. Tissue is removed using a sterile procedure, and the cells are dissociated using any method known in the art including treatment with enzymes such as trypsin, collagenase, and the like, or by using physical methods of dissociation such as with a blunt instrument.

Dissociation of cells can be carried out in any acceptable medium, including tissue culture medium. For example, a preferred medium for the dissociation of neural stem cells is low calcium artificial cerebrospinal fluid. The dissociated stem cells can be placed into any known culture medium capable of supporting cell growth, including HEM, DMEM, RPMI, F-12, and the like, containing supplements which are required for cellular metabolism such as glutamine and other amino acids, vitamins, minerals and useful proteins such as transferrin and the like. Medium may also contain antibiotics to prevent contamination with yeast, bacteria and fungi such as penicillin, streptomycin, gentamicin and the like. In some cases, the medium may contain serum derived from bovine, equine, chicken and the like. Serum can contain xanthine, hypoxanthine, or other compounds which enhance guanine nucleotide biosynthesis, although generally at levels below the effective concentration to suppress asymmetric cell kinetics. Thus, preferably a defined, serum-free culture medium is used, as serum contains unknown components (i.e. is undefined). Preferably, if serum is used, it has been dialyzed to remove rGNPrs. A defined culture medium is also preferred if the cells are to be used for transplantation purposes. A particularly preferable culture medium is a defined culture medium comprising a mixture of DMEM, F12, and a defined hormone and salt mixture. As indicated herein, by including a compound such as a rGNPr, asymmetric cell kinetics are suppressed. Thus, the effect of division by differentiated transit cells, which results in the diluting of the stem cells, is reduced.

The culture medium can be supplemented with a proliferation-inducing growth factor(s). As used herein, the term “growth factor” refers to a protein, peptide or other molecule having a growth, proliferative, differentiative, or trophic effect on neural stem cells and/or neural stem cell progeny. Growth factors that may be used include any trophic factor that allows stem cells to proliferate, including any molecule that binds to a receptor on the surface of the cell to exert a trophic, or growth-inducing effect on the cell. Preferred proliferation-inducing growth factors include EGF, amphiregulin, acidic fibroblast growth factor (aFGF or FGF-1), basic fibroblast growth factor (bFGF or FGF-2), transforming growth factor alpha (TGF.alpha.), and combinations thereof. Growth factors are usually added to the culture medium at concentrations ranging between about 1 fg/ml to 1 mg/ml. Concentrations between about 1 to 100 ng/ml are usually sufficient. Simple titration experiments can be easily performed to determine the optimal concentration of a particular growth factor.

In addition to proliferation-inducing growth factors, other growth factors may be added to the culture medium that influence proliferation and differentiation of the cells including NGF, platelet-derived growth factor (PDGF), thyrotropin releasing hormone (TRH), transforming growth factor betas (TGF.beta.s), insulin-like growth factor (IGF.sub.-1) and the like.

Stem cells can be cultured in suspension or on a fixed substrate. One particularly preferred substrate is a hydrogel, such as a peptide hydrogel, as described below. However, certain substrates tend to induce differentiation of certain stem cells. Thus, suspension cultures are preferable for such stem cell populations. Cell suspensions can be seeded in any receptacle capable of sustaining cells, particularly culture flasks, cultures plates, or roller bottles, more particularly in small culture flasks such as 25 cm² cultures flasks. In one preferred embodiment, cells are cultured at high cell density to promote the suppression of asymmetric cell kinetics.

Conditions for culturing should be close to physiological conditions. The pH of the culture medium should be close to physiological pH, preferably between pH 6-8, more preferably between about pH 7 to 7.8, with pH 7.4 being most preferred. Physiological temperatures range between about 30.degree. C. to 40.degree. C. Cells are preferably cultured at temperatures between about 32.degree. C. to about 38.degree. C., and more preferably between about 35.degree. C. to about 37.degree. C.

Cells are preferably cultured for 3-30 days, preferably at least about 7 days, more preferably at least 10 days, still more preferably at least about 14 days. Cells can be cultured substantially longer. They can also be frozen using known methods such as cryopreservation, and thawed and used as needed.

Another preferred embodiment provides for deriving clonal lines of somatic tissue stem cells by limiting dilution plating or single cell sorting. Methods for deriving clonal cell lines are well known in the art and are described for example in Puck et al., 1956; Nias et al., 1965; and Leong et al., 1985.

Uses of Expanded Somatic Stem Cells

The present invention also provides for the administration of expanded populations of stem cells to a patient in need thereof. The expanded stem cells of the present invention can be used for a variety of purposes, including but not limited to bone marrow transplants, gene therapies, tissue engineering, and in vitro organogenesis. Production of autologous stem cells to replace injured tissue would also reduce the need for immune suppression interventions. Preferred tissues for the isolation and expansion of somatic stem cells, for administration to a patient in need thereof, include but are not limited to the following: bone marrow, liver, lung, small intestine, colon, skin and cartilage such as from the knee. For example, somatic stem cells expanded from the skin may be used to generate new tissue for use in skin grafts.

Gene Therapy Applications

According to the invention, in addition to the introduction of genes that lead to constitutive upregulation of guanine ribonucleotides, the somatic stem cells can be further genetically altered prior to reintroducing the cells into the individual for gene therapy, to introduce a gene whose expression has therapeutic effect on the individual.

In some aspects of the invention, individuals can be treated by supplementing, augmenting and/or replacing defective and/or damaged cells with cells that express a therapeutic gene. The cells may be derived from stem cells of a normal matched donor or stem cells from the individual to be treated (i.e., autologous). By introducing normal genes in expressible form, individuals suffering from such a deficiency can be provided the means to compensate for genetic defects and eliminate, alleviate or reduce some or all of the symptoms.

A vector can be used for expression of the transgene encoding a desired wild type hormone or a gene encoding a desired mutant hormone. Preferably, as described above, the transgene is operably linked to regulatory sequences required to achieve expression of the gene in the stem cell or the cells that arise from the stem cells after they are infused into an individual. Such regulatory sequences include a promoter and a polyadenylation signal. The vector can contain any additional features compatible with expression in stem cells or their progeny, including for example selectable markers.

In another preferred embodiment, the transgene can be designed to induce selective cell death of the stem cells in certain contexts. In one example, the stem cells can be provided with a “killer gene” under the control of a tissue-specific promoter such that any stem cells which differentiate into cell types other than the desired cell type will be selectively destroyed. In this example, the killer gene would be under the control of a promoter whose expression did not overlap with the tissue-specific promoter.

Alternatively, the killer gene is under the control of an inducible promoter that would ensure that the killer gene is silent in patients unless the hormone replacement therapy is to be stopped. To stop the therapy, a pharmacological agent is added that induces expression of the killer gene, resulting in the death of all cells derived from the initial stem cells.

In another embodiment, the stem cells are provided with genes that encode a receptor that can be specifically targeted with a cytotoxic agent. An expressible form of a gene that can be used to induce selective cell death can be introduced into the cells. In such a system, cells expressing the protein encoded by the gene are susceptible to targeted killing under specific conditions or in the presence or absence of specific agents. For example, an expressible form of a herpes virus thymidine kinase (herpes tk) gene can be introduced into the cells and used to induce selective cell death. When the exogenous genetic material that includes (herpes tk) gene is introduced into the individual, herpes tk will be produced. If it is desirable or necessary to kill the transplanted cells, the drug ganciclovir can be administered to the individual and that drug will cause the selective killing of any cell producing herpes tk. Thus, a system can be provided which allows for the selective destruction of transplanted cells.

Administration of Expanded Somatic Stem Cells

This method involves administering by standard means, such as intravenous infusion or mucosal injection, the expanded stem cells to a patient.

The discovery that isolated stem cells may be expanded ex vivo and administered intravenously provides the means for systemic administration. For example, bone marrow-derived stem cells may be isolated with relative ease and the isolated cells may be cultured according to methods of the present invention to increase the number of cells available. Intravenous administration also affords ease, convenience and comfort at higher levels than other modes of administration. In certain applications, systemic administration by intravenous infusion is more effective overall. In a preferred embodiment, the stem cells are administered to an individual by infusion into the superior mesenteric artery or celiac artery. The stem cells may also be delivered locally by irrigation down the recipient's airway or by direct injection into the mucosa of the intestine.

After isolating the stem cells, the cells can be administered after a period of time sufficient to allow them to convert from asymmetric cell kinetics to exponential kinetics, typically after they have been cultured from 1 day to over a year. Preferably the cells are cultured for 3-30 days, more preferably 4-14 days, most preferably at least 7 days.

In one embodiment of the invention, the stem cells can be induced to differentiate following expansion in vitro, prior to administration to the individual. Preferably, the pool of guanine ribonucleotides is decreased at the same time differentiation is induced, for example by removal of the rGNPr from the culture medium (if a pharmacological approach has been used) or by downregulating expression of the transgene.

Differentiation of the stem cells can be induced by any method known in the art which activates the cascade of biological events which lead to growth, which include the liberation of inositol triphosphate and intracellular Ca.sup.2+, liberation of diacyl glycerol and the activation of protein kinase C and other cellular kinases, and the like. Treatment with phorbol esters, differentiation-inducing growth factors and other chemical signals can induce differentiation. Differentiation can also be induced by plating the cells on a fixed substrate such as flasks, plates, or coverslips coated with an ionically charged surface such as poly-L-lysine and poly-L-ornithine and the like.

Other substrates may be used to induce differentiation such as collagen, fibronectin, laminin, MATRIGEL.TM.(Collaborative Research), and the like. Differentiation can also be induced by leaving the cells in suspension in the presence of a proliferation-inducing growth factor, without reinitiation of proliferation.

A preferred method for inducing differentiation of certain stem cells comprises culturing the cells on a fixed substrate in a culture medium that is free of the proliferation-inducing growth factor. After removal of the proliferation-inducing growth factor, the cells adhere to the substrate (e.g. poly-omithine-treated plastic or glass), flatten, and begin to differentiate into neurons and glial cells. At this stage the culture medium may contain serum such as 0.5-1.0% fetal bovine serum (FBS). However, for certain uses, if defined conditions are required, serum would not be used.

Differentiation can be determined using immunocytochemistry techniques well known in the art. Immunocytochemistry (e.g. dual-label immunofluorescence and immunoperoxidase methods) utilizes antibodies that detect cell proteins to distinguish the cellular characteristics or phenotypic properties of differentiated cell types compared to markers present on stem cells.

For administration of stem cells, the isolated stem cells are removed from culture dishes, washed with saline, centrifuged to a pellet and resuspended in a glucose solution which is infused into the patient.

Between 10⁵ and 10¹³ cells per 100 kg person are administered per infusion. Preferably, between about 1-5×10⁸ and 1-5×10¹² cells are infused intravenously per 100 kg person. More preferably, between about 1×10⁹ and 5×10¹¹ cells are infused intravenously per 100 kg person. For example, dosages such as 4×10⁹ cells per 100 kg person and 2×10¹¹ cells can be infused per 100 kg person. The cells can also be injected directly into the intestinal mucosa through an endoscope.

In some embodiments, a single administration of cells is provided. In other embodiments, multiple administrations are used. Multiple administrations can be provided over periodic time periods such as an initial treatment regime of 3-7 consecutive days, and then repeated at other times.

One embodiment of the invention provides transgenic non-human animals into whose genome is stably integrated an exogenous DNA sequence comprising a constitutive promoter expressed in all cell types operably linked to a DNA sequence encoding a protein that leads to constitutive upregulation of guanine nucleotides, including the gene encoding inosine-5′-monophosphate dehydrogenase (IMPDH) or xanthine phophoribosyl transferase (XPRT). Preferably, the transgene is XPRT. Preferably, the transgenic animal is a mammal such as a mouse, rat or sheep.

The term “animal” here denotes all mammalian animals except humans. It also includes an individual animal in all stages of development, including embryonic and fetal stages. A “transgenic” animal is any animal containing cells that bear genetic information received, directly or indirectly, by deliberate genetic manipulation at the subcellular level, such as by microinjection or infection with recombinant virus.

“Transgenic” in the present context does not encompass classical crossbreeding or in vitro fertilization, but rather denotes animals in which one or more cells receive a recombinant DNA molecule. Although it is highly preferred that this molecule be integrated within the animal's chromosomes, the invention also encompasses the use of extrachromosomally replicating DNA sequences, such as might be engineered into yeast artificial chromosomes.

The term “germ cell line transgenic animal” refers to a transgenic animal in which the genetic information has been taken up and incorporated into a germ line cell, therefore conferring the ability to transfer the information to offspring. If such offspring, in fact, possess some or all of that information, then they, too, are transgenic animals.

The information to be introduced into the animal is preferably foreign to the species of animal to which the recipient belongs (i.e., “heterologous”), but the information may also be foreign only to the particular individual recipient, or genetic information already possessed by the recipient. In the last case, the introduced gene may be differently expressed than is the native gene.

The transgenic animals of this invention are other than human, and produce milk, blood serum, and urine. Farm animals (pigs, goats, sheep, cows, horses, rabbits and the like), rodents (such as mice), and domestic pets (for example, cats and dogs) are included in the scope of this invention. One preferred animal is a mouse. Mouse strains which are suitable for the derivation of transgenic mice as described herein are any common laboratory mouse strain. Preferred mouse strains to use for the derivation of transgenic mice founders of the present invention include FVB and C57 strains. Preferably, founder mice are bred onto wild-type mice to create lines of transgenic mice.

It is highly preferred that a transgenic animal of the present invention be produced by introducing into single cell embryos appropriate polynucleotides that encode XPRT or IMPDH, or fragments or modified products thereof, in a manner such that these polynucleotides are stably integrated into the DNA of germ line cells of the mature animal, and are inherited in normal mendelian fashion.

Advances in technologies for embryo micromanipulation now permit introduction of heterologous DNA into fertilized mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals. Those techniques as well known. See reviews of standard laboratory procedures for microinjection of heterologous DNAs into mammalian fertilized ova, including Hogan et al., MANIPULATING THE MOUSE EMBRYO, (Cold Spring Harbor Press 1986); Krimpenfort et al., Bio/Technology 9:844 (1991); Palmiter et al., Cell, 41: 343 (1985); Kraemer et al., GENETIC MANIPULATION OF THE EARLY MAMMALIAN EMBRYO, (Cold Spring Harbor Laboratory Press 1985); Hammer et al., Nature, 315: 680 (1985); Wagner et al., U.S. Pat. No. 5,175,385; Krimpenfort et al., U.S. Pat. No. 5,175,384, the respective contents of which are incorporated by reference. See also U.S. Pat. Nos. 4,736,866, 5,387,742, 5,545,806, 5,487,992, 5,489,742, 5,530,177, 5,523,226, 5,489,743, 5,434,340, and 5,530,179.

### EXAMPLES

### Example 1

### Derivation of Liver-Derived Stem Cells by Suppression of Asymmetric Cell Kinetics (SACK)

Using xanthosine (Xs) as a pharmacological agent to switch tissue stem cells from their default asymmetric cell kinetics program to exponential kinetics, we attempted to derive clonal rat liver epithelial stem cell lines that retained the ability to divide with asymmetric cell kinetics when Xs was removed. Rat liver epithelial cells found in the low-speed supernatant of centrifuged cells from in situ collagenase-perfused livers were clonally-derived by limiting dilution in 96-well plates in the absence or presence of added Xs (200 μM or 400 μM). Colonies with epithelial cell morphology arose with similar efficiency under both conditions (4.1±0.31 percent for both 480 Xs− wells and 960 Xs+ wells). The efficiency of establishing cell strains from these initial colonies was also similar, 50% (3/6) for colonies expanded in Xs-free medium and 65% (11/17) for colonies derived in Xs-containing medium. Each type of cell strain has now been maintained continuously in culture for >80 cell doublings. Early passage cells were cryo-preserved in liquid nitrogen and can be re-established in culture after thawing.

Three Xs-free (“Xs-F”) cell strains and 8 Xs-derived (“Xs-D”) cell strains were chosen for cell kinetics analysis. Despite the similarities in their derivation course, there is a remarkable difference in the growth properties of Xs-F and Xs-D cell strains (see Table 1). In colony formation assays in Xs-free medium, as a group, Xs-D cell strains exhibit poor growth compared to Xs-F strains. This is a highly significance difference (p=0.004). More importantly, in Xs-containing medium, on average their growth is not significantly different than that of Xs-F cell strains (p=0.08). The estimated mean increase in colony formation efficiency for Xs-D strains in Xs-containing medium is 11-fold, ranging from a 10% (Xs-D9; not statistically significant) to a 40-fold increase (Xs-D13; p<0.0001). The growth of Xs-F strains was unaffected by Xs even at the higher concentration of 100 μM (data not shown).

In only one case does the decreased growth of a Xs-D strain in the absence of Xs appear to be due to cell death (Xs-D7). After 2 weeks of culture in the absence of Xs, compared to their Xs-containing wells, strains Xs-D8, -D13, and -D15 show a significant reduction in colonies detected by visual inspection. However, when these same wells are examined by microscopy, a similar number of crystal violet-positive (i.e., viable) colonies are observed. This difference in quantitative results is due to the presence of micro-colonies with less than 20 cells that are not detected on simple visual inspection. Micro-colonies of this type are diagnostic of in vitro asymmetric cell kinetics (Sherley et al., 1995ab; Liu et al., 1998b). Since no micro-colonies are found in Xs-free plates of strain Xs-D7, it is likely that these cells die in the absence of Xs. This phenotype may indicate a cell variant with a defect in IMPDH, which is essential for growth, or a related function in guanine nucleotide metabolism. However, this explanation cannot account for the Xs-dependent colony formation of the other cell lines.

TABLE 1
Colony formation efficiency of rat liver
epithelial cell strains¹

| | | | |
|---|---|---|---|
| Cell Strain | Xs− | Xs+ | Xs+/Xs− |

| | |
|---|---|
| (Mean % ± SD; n = 6) | Ratio |

Derived in Xs− Medium

| | | | |
|---|---|---|---|
| Xs-F1 | 11 ± 3.4 | 9.8 ± 2.2 | 0.9 |
| Xs-F2 | 28 ± 2.6 | 29 ± 3.7 | 1.0 |
| Xs-F3 | 18 ± 2.8 | 18 ± 3.7 | 1.0 |

Derived in Xs+ Medium

| Xs-D4 | 5.2 ± 2.4 | 8.0 ± 1.1 | 1.5 |
|---|---|---|---|
| Xs-D6 | 3.9 ± 1.6 | 5.3 ± 1.8 | 1.4 |
| Xs-D7 | 0.0 ± 0.0 | 6.7 ± 2.2 | ≧22 |
| Xs-D8 | 1.3 ± 0.53 | 22 ± 3.6 | 17 |
| Xs-D9 | 9.7 ± 3.4 | 11 ± 2.6 | 1.1 |
| Xs-D13 | 0.3 ± 0.26 | 12 ± 2.7 | 40 |
| Xs-D14 | 0.3 ± 0.42 | 0.4 ± 0.67 | 1.3 |
| Xs-D15 | 1.9 ± 1.4 | 10 ± 1.6 | 5.3 |
| ¹Each cell strain was plated at 200 cells per each well of two 6-well plates in their respective culture medium used for propagation. After 16 hours, one plate was replaced with X-free medium (Xs−) and the other plate was replaced with medium containing 50 μM xanthosine (Xs+). Plates were incubated for 14 | | | |
| # days and then fixed and stained with crystal violet. Colonies detected by visual inspection were counted to determine the colony formation efficiency ([colony number/200] × 100%). Data are presented as the mean % ± standard deviation of replicate wells. | | | |

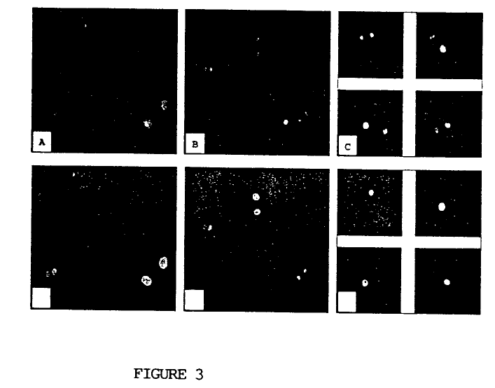

Although line Xs-D13 had a greater Xs+/Xs− colony formation efficiency ratio (see Table 1), Xs-D8 cells showed better growth under both culture conditions. We developed a new assay to directly visualize Xs-D8 asymmetric daughter cells based on the observation that arrested daughters do not synthesize DNA (Sherley et al., 1995a). The assay is called a “daughter pair analysis” (DPA). To perform the DPA, cells are plated in Xs-containing medium at microcolony density (approximately one cell per 40× microscope field). After allowing 4 to 5 hours for cell adherence, asymmetric cell kinetics are induced by changing to Xs-free medium. After about 16 hours to allow cell divisions to produce “daughter pairs”, the cells are labeled for 4 hours with the thymidine analogue bromodeoxyuridine (BrdU). The cells are then fixed and evaluated for BrdU incorporation by in situ immunofluorescence. FIG. 3 shows results for line Xs-D8 and, as a control, line Xs-F1, derived by conventional means in the absence of Xs.

When grown in the absence of Xs, 84% of Xs-D8 BrdU(+) daughter pairs show a single BrdU(+) daughter (n=76 pairs; e.g., FIGS. 3C,F). The BrdU(+) daughter is the cycling stem cell daughter, whereas the BrdU(−) daughter corresponds to a non-cycling transit cell daughter that can undergo differentiation in peptide hydrogels (data not shown). Consistent with its ability to shift cells from asymmetric to exponential kinetics, addition of Xs increases the fraction of double-BrdU(+) Xs-D8 daughter pairs from 16% to 76% (n=58 pairs; e.g., FIGS. 3B,E). In contrast to Xs-D8 cells, the majority of Xs-F1 BrdU(+) daughter pairs in Xs-free medium were symmetric (FIGS. 3A,D), consistent with Xs-independent exponential cell kinetics. For 70 scored daughter pairs in Xs-free medium, the frequency of Xs-F1 symmetric pairs was 5 times the frequency of Xs-D8 symmetric pairs.

Hepatocyte Differentiation Properties of Xs-D Cell Lines in Adherent Culture

The kinetics analyses described above support our proposal that cells with conditional asymmetric cell kinetics can be derived from adult somatic tissues by suppressing their inherent asymmetric kinetics with Xs, a guanine nucleotide pool expander (Merok and Sherley, 2001). We next demonstrated that several of the Xs-D lines had differentiation properties consistent with being derived from hepatocyte stem cells. Our approach in these studies was to evaluate different strategies that might induce Xs-D cells to become functional hepatocytes in vitro. Because of the importance of cell growth arrest for cell differentiation in vivo, we initially focused on approaches that would induce growth cessation.

In vivo, cell differentiation in somatic tissues has two important cell kinetics features. First, it occurs after a fixed number of cell divisions from the stem cell stage; and second, cells undergo a cell kinetics arrest before terminal differentiation occurs (Potten and Morris, 1988). Ideally, we wanted to mimic this process in culture with cells dividing with asymmetric stem cell kinetics. However, there was concern whether in vitro asymmetric divisions would be in appropriate balance with non-dividing cells. So, we decided to augment the growth arrest by growing cells to confluency in either Xs-free or Xs-containing medium and then inducing a total quiescent state by reducing the serum concentration to 1%. Many in vitro cell models have been described that undergo differentiation upon serum reduction.

In summary, we found that under conditions of active growth or quiescence, with one exception, the Xs-F and Xs-D lines do not express markers for non-hepatocyte liver cell types (i.e., bile epithelial cells, Kupffer cells, or stellate cells). The marker we initially selected as an endothelial cell-specific marker, ICAM-1, turned out to recognize all cell types in non-parenchymal liver cell fractions (data not shown). In the future, analyses for desmin expression will be performed to confirm that Xs-F and Xs-D lines are not of endothelial origin. Given the great difficulty in culturing liver endothelial cells and later finding albumin expression in several of the lines (see below), such a cell type origin is very unlikely.

We examined α-fetoprotein expression as an indicator of primitive cell properties characteristic of stem cells. Only Xs-D lines showed any expression of this protein. Quiescent Xs-D9 cells consistently showed weak expression, and the expression was Xs-independent. In an analysis under conditions of quiescence developed in glucose-free medium, Xs-D13 showed a significant fraction of α-fetoprotein-positive cells. More importantly, these cells occur specifically in the absence of Xs, the condition that supports asymmetric cell kinetics. Glucose withdrawal has been shown to induce differentiation in tumor cell lines derived from gastrointestinal tissues (Neutra and Louvard, 1989). Thus, the finding of α-fetoprotein may be relevant to tissue differentiation mechanisms in vivo. Further investigation of cells under glucose-free conditions will be required to determine the significance of α-fetoprotein induction in Xs-D13 cells. Xs-D8 cells have not been examined for α-fetoprotein expression under these conditions.

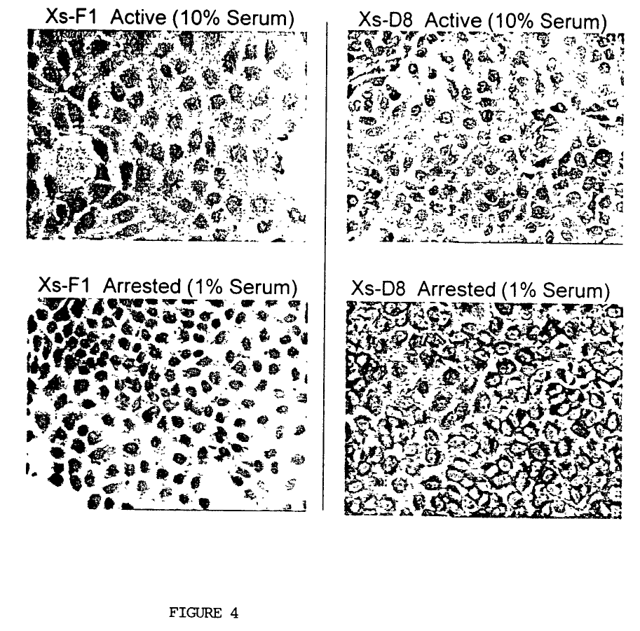

The most dramatic differentiation result obtained in the adherent culture studies was the induction of albumin expression in Xs-D lines under conditions of quiescence (See example in FIG. 4). Line Xs-F1 also showed a small degree of albumin induction, but it was quite low compared to the Xs-D lines (FIGS. 4C and D, respectively). Three other treatments were added to the standard quiescence-induction protocol to evaluate their effect on albumin induction. These were DMSO to mimic glucocorticoid effects, growth on collagen as a form of extracellular matrix, and glucose-removal as noted above for its differentiation effects on gastrointestinal cell lines. Whereas DMSO had no noticeable effect on the degree of albumin expression, collagen and glucose-removal reduced and prevented the induction of albumin, respectively. This effect of glucose on albumin production is consistent with its induction of more primitive cells that express α-fetoprotein.

Lines Xs-F1, Xs-F3, Xs-D8, and Xs-D13 lines have been evaluated for expression of H4 antigen, a mature hepatocyte marker (Petersen et al., 1999) from Dr. Doug Hixon's lab at Brown University in Providence, R.I. All four lines exhibit expression of H4 under conditions of cell growth arrest in 1% serum. The greatest expression was seen in line Xs-D8 under conditions of arrest in xanthosine-free medium (data not shown). H4 expression under conditions of active cell growth has not been evaluated.

In addition to differences in expression of differentiation markers, the Xs-D lines are morphologically distinct from Xs-F lines. Xs-F cells maintain a similar homogenous field of cells under conditions of active growth or quiescence. In contrast, Xs-D cells show a heterogeneous field of cells that becomes highly contoured after 2 weeks under conditions of quiescence (data not shown). Interestingly, although quiescent Xs-D cells appear highly compact and small, these cell layers produce a unique type of large cell that is found in suspension. These large cells can be re-plated and cultured on collagen coated dishes. They appear to be distinct in morphology from the cells in their culture of origin, and most are binucleated (data not shown). Multinucleated hepatocytes are commonly observed in the liver parenchyma in vivo.

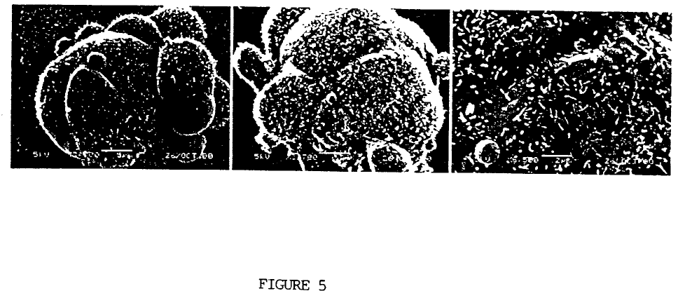

Xs-D8 cells appeared to be the Xs-derived line with the most complete hepatocyte differentiation properties. When Xs-F1, Xs-F3, Xs-D8, and Xs-D13 were evaluated for the ability to form spheroid aggregates in spinner culture, only Xs-D8 and Xs-D13 formed spheroids of significant size, with Xs-D8 being clearly superior (data not shown). Spheroid formation is another well-described property of mature hepatocytes in culture (Powers et al., 1997; Mitaka et al., 1999). It is particularly noteworthy that Xs-D8 and Xs-D13 are the lines with the most pronounced Xs-dependent growth phenotype. Scanning electron microscopy analysis of Xs-D8 spheroids show two features indicative of differentiation, morphological heterogeneity and surface structures indicative of microvilli formation (see FIG. 5)

Confirmation that the surface projections are microvilli will be confirmed by transmission electron microscopy detection of cytoskeletal components in the structures. Xs-D8 cells are also being evaluated for evidence of bile secretion properties when differentiated in Matrigel™ with 1% serum. Under this condition, Xs-D8 cells exhibit canaliculi-like intercellular spaces that are visible by phase microscopy (data not shown).

These data support our teaching that somatic stem cells can be propagated in culture by regulation of their cell kinetics. In the very first attempt, it turned out to be quite straightforward to derive several rat liver epithelial cell lines with conditional asymmetric cell kinetics. This result has importance in liver cell biology in general. The question of whether liver contains typical stem cells has long been a highly controversial issue. The controversy arises because mature hepatocytes are known to have remarkable regenerative capability after liver injury. Thus, the need to invoke a steady state stem cell function has been questioned. However, it is clear that a low level of continuous cell division and apoptosis occurs in normal liver. The cellular basis for this turnover activity is unknown but highly debated (Grisham and Thorgeirsson, 1997). The extraction of rare cells from the liver with asymmetric cell kinetics supports the idea that typical stem cells do exist in the liver. We estimate that the in vivo progenitors of Xs-D lines occur at roughly the same frequency as rare cycling liver cells.

The Xs-D lines do have several properties consistent with being derivative of stem cells or potential stem cells. Moreover, Xs-F lines share only a few of these traits. It would not be surprising, if we fail to re-create the conditions in vitro that are necessary to promote their differentiation in vivo. However, a positive control cell that retains hepatocyte differentiation properties in vitro as a standard for comparison is still desired.

### Example 2

### SACK Effects on Non-Adherent Cell Production in Long-Term Murine Bone Marrow Cultures

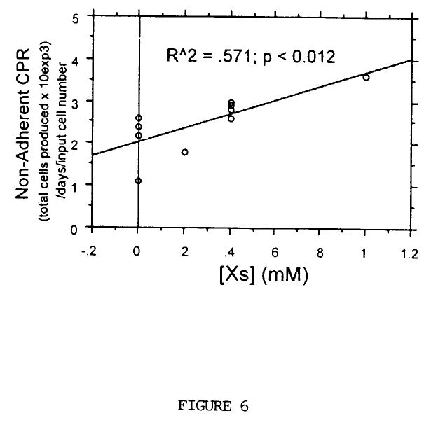

To develop long-term bone marrow cultures, freshly isolated bone marrow cells from tibia and femurs of 7-12 week-old female C57BL/6J mice were grown in culture medium supplemented with 25% horse serum. As shown in FIG. 6, supplementation of culture medium with the SACK agent Xs (1 mM) resulted in an 80% increase in the rate of production of non-adherent, differentiated cells.

Detection of Long-Term Colony Forming Cells (LT-CFCs) in Soft Agar

Because of the difficulties anticipated in cloning Xs-dependent cells from heterogenous long-term bone marrow cultures, we decided to adopt a soft agar culture strategy. It is well known that hematopoietic progenitor cells will form colonies when grown in semi-solid medium (Dexter et al., 1984). This property does not mean that they are transformed like tumor cells. Instead, it simply reflects their natural ability to divide without anchorage. In many bone marrow cell studies, conditions are selected to induce bone marrow cell differentiation. Methyl-cellulose or soft agar supplemented with lineage-promoting growth factors are widely used experimental systems (Dexter et al., 1984). For ideal SACK, we wished to minimize stimuli for differentiation. Therefore, we used agar developed in routine horse serum-supplemented long-term culture medium, without any additional growth factor source, as the gel medium for our studies. We followed procedures developed for detecting anchorage-independent growth of transformed tumor cells.

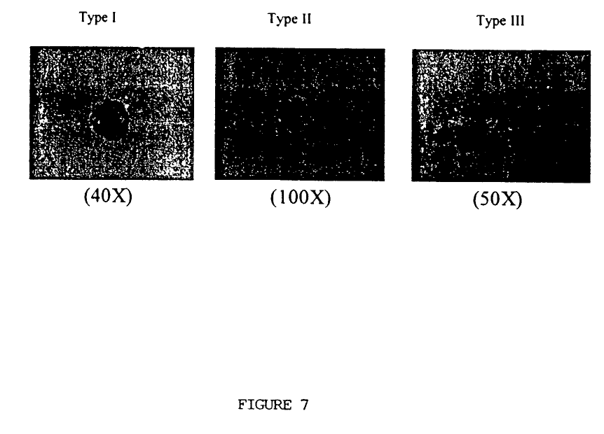

When fresh bone marrow is plated in soft-agar under these conditions, 1-2 weeks later cell colonies are detected. The rapid nature of this assay greatly improved our experimental throughput. The evaluation of effects in long-term cultures takes 2-3 months to complete. Three colony morphologies are detected. Examples of these, designated type I, type II, and type III, are shown in FIGS. 7A-C. Type I colonies appear less differentiated than type II and III colonies.

In prior studies of bone marrow colony formation in soft agar, several types of common colonies have been described (Dexter et al., 1984). Many of these are detected after stimulation of their growth by specific growth factors or conditioned medium from cultured cell lines or activated splenocytes. Though morphological similarities are notable between our type II and type III colonies and those described by others (e.g., granulocyte and macrophage colonies), thus far in our literature review, our type I colonies' morphology appears unique.

It has been shown that LT-CFCs, which persist in long-term bone marrow cultures, possess bone marrow reconstitution activity (Cheshier et al., 1999). Our plan was to evaluate SACK effects on soft agar colonies derived from fresh bone marrow. Therefore, we wished to relate the morphology of colonies that arose from fresh bone marrow cells to those that arose from LT-CFCs in long-term cultures. We examined control (Xs-free medium) long-term bone marrow cultures for the presence of cells that form colonies in soft agar. After 13 weeks of culture, we found that non-adherent cells in culture supernatants produced only type II and III colonies. These colonies arose at a frequency of 5×10⁻⁵. After 9 weeks of culture, when adherent cells were evaluated after washing and then removal with half-strength trypsin, only type II colonies were detected (frequency=19×10⁻⁵). However at 11 and 15 weeks, adherent cells yielded only type I colonies (frequency=7.8×10⁻⁵). Adherent cells from cultures with Xs-containing medium (0.4 to 1.0 mM Xs) also produced only type I colonies. Paralleling the modest increase observed for their non-adherent cell production rate, adherent cells from Xs-containing cultures yielded on average 35% more type I colonies than control cultures. The persistence and adherence of cells that produce type I colonies identified them as LT-CFCs. The finding that cells that produced type II and type III colonies were found primarily in long-term culture supernatants identified them as short-term differentiating progenitor cells.

SACK Effects LT-CFC Kinetics

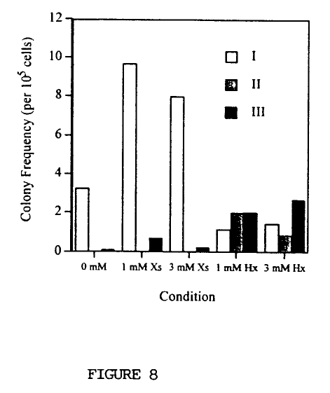

Given their associated HSC activity, LT-CFCs are predicted to divide with asymmetric cell kinetics. We evaluated whether Xs would increase the frequency of LT-CFC type I colonies in soft agar. Freshly prepared bone marrow cells were plated in soft agar developed in either control medium or medium containing 1 mM or 3 mM Xs. As shown in FIG. 8, the mean fold increase in type I colony formation associated with Xs addition (1 mM and 3 mM) was 2.9 (range 1.2 to 6.2; p<0.017). Only type I colonies exhibited a significant increase in frequency. In contrast, hypoxanthine (Hx), a related purine base not previously evaluated for SACK activity, increased the frequency of type II and III colonies at the expense of type I colonies. In published studies, we have shown that Hx is not as effective a SACK agent as Xs (Sherley, 1991). Thus, we interpret the results with Hx, in part, to reflect less effective SACK of LT-CFC resulting in higher frequencies of differentiating progeny of asymmetric cell divisions. Of course, we cannot exclude the possibility that Hx simply promotes type I and type II cell production per se. Future experiments in which both Hx and Xs are added together may shed light on this question. The effects of Hx were consistent with the predicted precursor:progeny relationship between LT-CFCs and short-term differentiating progenitor cells that produce type II and III colonies.

At a density of 1×10⁵ bone marrow cells per 6-well, addition of 1 mM Xs resulted in a 1.5-fold increase in the frequency of type I colonies. However, in experiments with inputs of 5×10⁵ or 1×10⁶ cells, a mean fold increase of 5.3-fold was observed. In earlier studies, we showed that, in vitro, increased cell density is an important physiological factor that promotes a shift from asymmetric cell kinetics to exponential cells kinetics (Rambhatla et al., 2001). Thus, finding that Xs-induced type I colony formation is cell density-dependent supports our hypothesis that these colonies arise from LT-CFCs that normally divide with asymmetric cell kinetics.

Serial Transfer of LT-CFCs in Soft Agar

We next evaluated whether LT-CFCs which produced type I colonies could be propagated in soft agar. Pasteur pipettes were used to isolate type I colonies from soft agar developed in either control medium or medium containing Xs. Isolated colonies were dispersed and their cells replated in their entirety in a single well of a 6-well plate in soft agar developed with the respective Xs concentration. The overall efficiency of secondary colony formation was high (52%). It was noteworthy that in these experiments cases of multiple colonies per well (2-15) occurred frequently (32%). Eighty-three percent of the cases of multiple secondary colonies occurred in Xs-containing medium. In all but one case, multiple secondary colonies were of the same type. For all conditions, the mean number of secondary colonies produced per primary colony was 1.2±2.6. This result suggest that on average each type I colony contains one LT-CFC. The colony formation results demonstrate that propagation of LT-CFCs is promoted by Xs. The fact that the occurrence of multiple secondary colonies is 5 times more common in the presence of Xs indicates that Xs increases the frequency of symmetric divisions by LT-CFCs.

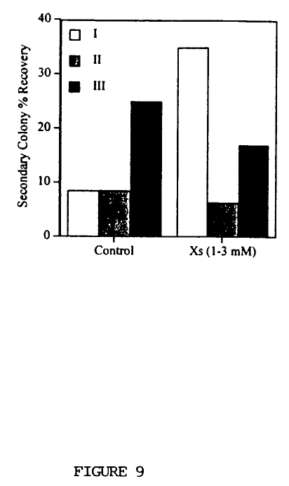

Cells from type I colonies grown in control soft agar gave rise to secondary colonies of all three types, with type II and type III colonies dominating 4:1 over type I colonies (see FIG. 9). This result supported our earlier conclusion that type II and type III colonies arise from short-term progenitor cells that are the progeny of LT-CFC divisions. However, to date, we have not evaluated whether type II and type III colonies can produce type I colonies. Being derivative of short-term differentiating progenitor cells, type II and III colonies are predicted to exhibit low secondary colony formation activity in general.

Type I colonies grown in Xs gave rise to type I colonies with 4-fold greater efficiency than control type I colonies (see FIG. 9). In contrast to control type I cells, cells from type I colonies grown in Xs produced predominantly type I colonies again. In Xs-containing soft agar, type I colonies occurred 50% more frequently that type II and III colonies. These results suggest that Xs increases the stability and propagation of LT-CFCs in culture. We postulate that this occurs by induction of symmetric LT-CFC divisions that increase LT-CFC number and reduce the rate of development of differentiating LT-CFC progeny cells.

### Example 3

### Regulation of Asymmetric Stem Cell Kinetics by IMP Dehydrogenase (IMPDH)

Regulation of the rate-limiting enzyme for guanine nucleotide metabolism, inosine-5′-monophosphate dehydrogenase (IMPDH), by the p53 tumor suppressor protein is required for asymmetric cell kinetics in murine cells. In silico interrogation of global gene expression data for the human type II IMPDH gene indicates that p53 is also an important determinant of IMPDH-dependent mechanisms that control cell kinetics in human cancer cells.

In vitro cell models for asymmetric cell kinetics can be used to define genes that control deterministic programs for asymmetric cell kinetics. Previously, we discovered that “growth-suppression” by the wild-type p53 tumor suppressor gene in cultured cells is due to the ability of p53 to switch individual cells from symmetric cell kinetics (i.e., all divisions producing two “stem cell daughters”) to deterministic asymmetric cell kinetics (Sherley et al. 1995ab, Rambhatla et al. 2001). Here, we show that the ability of p53 to induce this switch requires down-regulation of the rate-limiting enzyme for guanine nucleotide biosynthesis, inosine-5′-monophosphate dehydrogenase (IMPDH; EC 1.1.1.205).

IMPDH Regulation and Asymmetric Cell Kinetics

IMPDH is a ubiquitously expressed essential enzyme (Stadler et al. 1994, Senda et al. 1994, Gu et al., 2000). In human cells, two different single-copy genes express two highly homologous isoforms (type I and type II; 84% amino acid identity; Natsumeda et al. 1990). Whereas the type I gene shows little regulation with cell growth state, changes in the expression of the type II gene have been associated with cell proliferation, cell differentiation, and malignant transformation of rodent and human cells (Jackson et al. 1975, Nagai et al. 1992, Collart et al. 1992). The type II IMPDH is also highly conserved during evolution (Natsumeda et al. 1990). We have suggested that increased IMPDH gene expression indicates that cells have acquired the capacity for continuous exponential kinetics, as opposed to activation of cell division per se. Immortal cells in culture express high levels of IMPDH activity whether actively dividing or arrested by growth factor removal (Stadler et al. 1994).

Our p53-inducible model cells are the first mammalian cells demonstrated to divide with deterministic asymmetric cell kinetics (Sherley et al. 1995ab). The purine nucleoside xanthosine can suppress the asymmetric cell kinetics of these cells (Sherley et al. 1995a, Liu et al. 1998a). Xanthosine prevents the p53-dependent shift from exponential cell kinetics, typical of immortalized cells and tumor-derived cells, to asymmetric cell kinetics. Xanthosine is converted into xanthosine-5′-monophosphate, the product of the IMPDH reaction, in one step by ubiquitous nucleoside kinases (Kornberg 1980).

The ability of xanthosine to suppress asymmetric cell kinetics implicated IMPDH regulation as an important determinant of cell kinetics symmetry. Subsequently, we showed that down-regulation of IMPDH by wild-type p53 is required for p53-dependent growth suppression. In both murine epithelial cells and fibroblasts engineered for conditional p53 expression, cellular IMPDH mRNA, protein, and activity decline in response to near basal p53 expression (Liu et al. 1998a). Down-regulation of IMPDH activity and its guanine ribonucleotide products by p53 is required for the growth suppression that occurs when p53 expression is modestly elevated in epithelial cells with a wild-type p53 genotype or simply restored to basal levels in p53-null fibroblasts (Sherley et al. 1995a, Liu et al. 1998ab, Sherley 1991). This requirement was shown definitively in studies with isogenic p53-inducible cell lines that contain a constitutively expressed IMPDH transgene (Liu et al. 1998b). These “impd-transfectant” lines are isogenic to p53-inducible cells derived from p53-null murine embryo fibroblasts. They express basal levels of wild-type p53 protein in response to micromolar concentrations of zinc chloride, which activates the modified metallothionein promoter that controls expression of their p53 transgene. However, they maintain IMPDH expression at levels comparable to that of cells that do not express p53. Impd-transfectants show little or no p53-dependent growth suppression, even though they retain inducible wild-type p53 function (Liu et al. 1998b).

Growth suppression by p53 in the Zn-dependent fibroblast lines is due to the induction of asymmetric cell kinetics. Several cell kinetics assays, including time-lapse digital microscopy, were used to make this determination (Rambhatla et al. 2001). Failure to down-regulate IMPDH activity sufficiently in isogenic impd-transfectants might prevent “growth suppression” by one of two different mechanisms: 1) continued exponential cell kinetics; or 2) induction of asymmetric kinetics, but with production of cycling asymmetric daughters that have a significantly reduced cell cycle time. We used a bromodeoxyuridine-Hoechst dye quench (BrdU-HO quench) procedure (Latt et al. 1977) to distinguish between these two mechanisms.

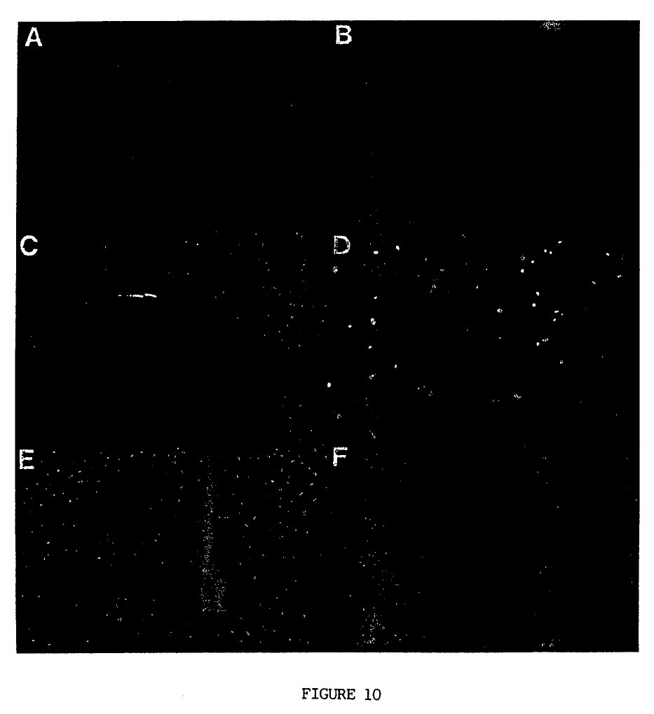

The BrdU-HO quench procedure is performed by culturing cells for one generation period with the thymidine analogue BrdU and then examining their UV-excited nuclear fluorescence after staining with Hoechst dyes. Because non-cycling daughters produced by asymmetric cell kinetics are non-replicative (Sherley et al. 1995a; data not shown), they are distinguished from cycling daughters by their failure to incorporate BrdU. When cycling cells, which incorporate BrdU during S phase, are stained with Hoechst dye, their nuclear fluorescence is quenched by the BrdU and appears dim compared to the bright fluorescence of nuclei in cells that have not incorporated BrdU. When cultured for at least one cell cycle period, all nuclei of cells dividing with exponential kinetics will uptake BrdU and therefore exhibit uniform dim nuclear HO fluorescence. Under control or p53-inducing condition, nuclei from p53-null control cells are uniformly dim (FIGS. 10A and 10B). Similarly reflecting their exponential cell kinetics, p53-inducible cells grown under non-inducing conditions exhibit uniformly dimly fluorescent nuclei (FIG. 10C).

In contrast, cells dividing with asymmetric cell kinetics (conditions of p53 expression; FIG. 10D) exhibit both dim nuclei and bright nuclei. The dim nuclei correspond to cycling asymmetric stem cell daughters, whereas the brightly fluorescent nuclei correspond to asymmetric non-cycling daughters. FIG. 10D also illustrates the overall “growth suppression” that is observed when cultured cells switch from exponential kinetics to asymmetric kinetics. By evaluating the %bright cell fraction as a function of the time and duration of BrdU labeling, the BrdU-HO quench method can be used to demonstrate and quantify asymmetric cell kinetics (data not shown). For several different independently derived p53-inducible cell lines, the mean % bright fraction after 48 hours of incubation in BrdU under conditions of p53 expression was 37±12% (n=5; p=0.002; Table 2 data). This experimental value is in good agreement with the expected value for ideal asymmetric cell kinetics of 40% after 48 hours of BrdU labeling (Sherley et al. 1995a, Rambhatla et al. 2001). Quantification of the % bright fraction by both fluorescence digital imaging and flow cytometry was used to confirm conclusions from fluorescence microphotography (data not shown). Compared to control p53-inducible vector-transfectants, impd-transfectants showed a marked reduction in the % bright cell fraction (Compare FIGS. 10D and 10F; Table 2, tC-lines versus tI-lines). This finding demonstrates that unless IMPDH is reduced below its basal level, p53 cannot initiate an asymmetric cell kinetics program.

Quantitative Effects of IMPDH on Cell Kinetics

We performed analyses to quantify the degree to which changes in p53 and IMPDH expression effect asymmetric cell kinetics. Previously, we developed a method to quantify asymmetric cell kinetics in terms of a mathematical parameter F_{d}, the fraction of new daughter cells that divide (Sherley et al. 1995b, Rambhatla et al. 2001). For asymmetric cell lineages, F_{d} approximates 0.5; whereas for exponential cell lineages, Fd approaches 1.0. On the time-scale of our experiments, individual cell lineages adopt discretely one or the other of these two cell kinetics programs (Sherley et al. 1995a, Rambhatla et al. 2001). Therefore, the average F_{d} of a cell population gives an estimate for P_{ack}, the probability that any cell in the population will initiate deterministic asymmetric cell kinetics.

We have described methods for determination of the average F_{d} of cultured cell populations (Sherley et al. 1995b). Theoretically, for the range of F_{d}=0.5 to 1.0, ΔP_{ack}/ΔF_{d}=−2. For exponentially dividing p53-null fibroblasts and asymmetrically dividing p53-expressing fibroblast, we independently determined F_{d} and estimated values for P_{ack} from published time-lapse data (Rambhatla et al. 2001). These data (F_{d}=0.79 and 0.49, respectively; and P_{ack}=0.09 and 0.72, respectively) yield −2.1 as the experimentally determined ΔP_{ack}/ΔF_{d}, in excellent agreement with the theoretical value, −2. Based on this determination, ΔP_{ack}/ΔF_{d}=−2.1 was used to estimate ΔP_{ack} from experimentally determined ΔF_{d} values.

With published data from the Zn-dependent model cells (Rambhatla et al. 2001), simple linear regression analyses were used to estimate independent rates of change in F_{d}, p53 protein, IMPDH protein, and IMPDH activity with respect to the common variable Zn concentration. Table 3 lists the rates determined and the level of statistical significance of each regression analysis. The chain rule of differentials was then applied to yield estimates of the dependency of P_{ack} on changes in p53 and IMPDH (see Table 3 for calculations). The results of this analysis show that the probability that cells will divide with asymmetric cell kinetics increases dramatically in response to small changes in p53 and IMPDH. The averaged absolute value of 12 for ΔP_{ack}/(Δp53 or ΔIMPDH) is consistent with our observations that a 30-50% reduction in IMPDH in response to modest levels of wild-type p53 precipitates a nearly quantitative shift from exponential to asymmetric cell kinetics (Sherley et al. 1995ab, Rambhatla et al. 2001).

In Silico Analysis of IMPDH in Cancer Cells

We performed a micro-array database analysis to investigate the significance of p53-IMPDH interactions in the growth regulation of human cancer cells. Simple linear regression analyses were used to look for statistically significant associations between type II IMPDH mRNA expression, p53 mRNA expression, cell doubling time, and p53 genotype in micro-array data extracted from the Stanford NCI60 database (Ross et al. 2000). This database contains normalized mRNA expression data for approximately 8,000 gene sequences for 60 cell lines derived from tumors of different human tissues.

No significant association was detected between wild-type p53 mRNA expression and type II IMPDH mRNA expression (n=20; R²=0.016; p=0.597). Because of the high degree of post-translational regulation that p53 is known to undergo (Giaccia and Kastan 1998), a relationship between its mRNA and regulated target genes may be difficult to detect. Consistent with this explanation, only a weak association was detected between wild-type p53 mRNA and p21waf1 mRNA (n=20; R²=0.117; p=0.031), a highly p53-induced mRNA (El-Deiry et al. 1993). Moreover, the regression coefficient from this analysis is negative (−1.15), a result that is contrary to the known positive correlation between wild-type p53 protein expression and p21waf1 mRNA level.

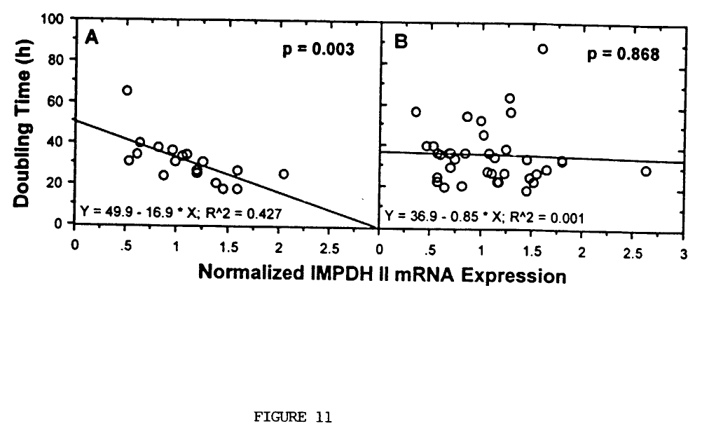

Although no association was detected between the expression level of p53 mRNA and type II IMPDH mRNA, p53 genotype had a profound effect on the association between type II IMPDH mRNA expression and cell proliferation rate. The developers of the NCI60 micro-array database have reported an association between type II IMPDH mRNA and cell doubling time (Ross et al. 2000). The association was detected in a cluster image map including data for all 60 cell lines; and the analysis was not stratified by p53 genotype. By simple regression analysis, we found that, in cells of wild-type p53 genotype, type II IMPDH mRNA showed a significant association with cell doubling time (n=18; R²=0.427; p=0.003; FIG. 11A). Cell lines with higher proliferative rate (i.e., shorter doubling time) exhibit higher levels of type II IMPDH mRNA expression. Our studies indicate that in murine cells with wild-type p53 protein expression, IMPDH mRNA, protein, and activity vary coordinately (Liu et al. 1998a); and the single murine form of IMPDH is most homologous to the human type II protein (Tiedeman and Smith 1991). Therefore, the observed association is consistent with the hypothesis that type II IMPDH functions as a rate determining factor for cell proliferation in human cancer cells that express wild-type p53.

In contrast to cells that express wild-type p53 protein, cell lines expressing mutant p53 proteins showed no significant association between type II IMPDH mRNA and cell doubling time (n=37; R²=0.001; p=0.868; FIG. 11B). Thus, wild-type p53 appears to be an important determinant of this relationship in human tumor cells (compare FIGS. 11A and 11B). The loss of the association may reflect deregulation or mal-regulation of the type II IMPDH gene due to loss of the input from normal p53 protein. Of course, all of the cells in this study have undergone neoplastic transformation. The available data do not allow us to determine whether each tumor line expresses a higher level of IMPDH than its tissue of origin. However, elevated expression of type II IMPDH in malignant cells is a well-described property of the enzyme (Collart et al. 1992).

It is noteworthy that the distributions of type II IMPDH mRNA levels in cells with wild-type p53 versus mutant p53 are quite similar (mean normalized expression level±standard deviation=1.1±0.39 versus 1.1±0.47, respectively). Because IMPDH is an essential enzyme, the finding of similar levels of expression in populations of either genotype may indicate a narrow range of elevated IMPDH expression that promotes tumor formation. In cells that retain wild-type p53 expression, such a requirement for IMPDH up-regulation for tumor growth must be accomplished by other mechanisms. The elucidation of such mechanisms is likely to reveal components of pathways that control cell kinetics programs in human tissue cells.

Presently, our knowledge of the detailed molecular functions of p53 and IMPDH in asymmetric cell kinetics control is limited. For example, although IMPDH down-regulation is required for asymmetric cell kinetics, whether it is sufficient has not been established. It may be that changes in the expression of other p53-regulated genes (e.g., the cyclin-dependent kinase inhibitor p21 waf1) are also required for p53-dependent asymmetric cell kinetics. Similarly, regulation of IMPDH may not occur via direct gene repression by p53, but indirectly via other p53-responsive factors.

Herein, we report the integration of original laboratory investigations with gene expression database interrogations to develop a more complete understanding of a newly defined cellular pathway that regulates deterministic asymmetric cell kinetics. The p53 tumor suppressor gene and an important nucleotide metabolism enzyme, IMPDH, are the first genetic components of the pathway to be described in mammalian cells. Only modest reduction in IMPDH expression, as a result of wild-type p53 expression, is required to maintain cultured murine cells in a state of asymmetric cell kinetics. The presented in silico (Maurer et al. 2000, Dearden and Akam 2000) gene expression database analyses extend the importance of p53-dependent IMPDH expression to cell kinetics control in human cancer cells. These analyses portend similar roles for p53 and IMPDH in mechanisms that regulate asymmetric stem cell kinetics in human somatic tissues.

Materials and Methods

Cell Culture

The culture conditions for all cell lines used in the study have been described in detail previously. Temperature-dependent p53-inducible line 1h-3 and paired control line 1g-1 were cultured as described by Sherley (1991). Zn-dependent p53-inducible fibroblasts (lines Ind-4 and Ind-8) and p53-null control fibroblasts (Con-3) were maintained as described by Liu and others (Liu et al. 1998ab). Zn-dependent, p53-inducible, impd-transfectant cells (tI-1, tI-3, and tI-5) and control vector-transfectant cells (tC-2 and tC-4) were maintained as previously described (Liu et al. 1998b).

BrdU-HO Quench Assay

After 24 hours of growth under control non-inducing conditions or p53-inducing conditions in plastic 6-well culture plates, BrdU was added (0.5 μM) and culture continued for an additional 48 hours. At the end of this period, cells were washed briefly in phosphate-buffered saline (PBS) warmed to 37° C., fixed for 15 minutes in absolute methanol chilled to −20° C., and then air-dried at room-temperature. Dried fixed cells were stained for 40 minutes in the dark at room temperature with 0.5 μg/ml Hoechst 33258 dissolved in PBS. After washing with PBS at room temperature, the cells were examined for UV-epifluorescence. Fluorescent nuclei were photographed with a 10× microscope objective.

Micrographs were used to determine the percent of cells with brightly fluorescent nuclei. One hundred to 200 cells were counted for epithelial cell line analyses; and at least 500 were counted for fibroblast lines. Cells with brightly fluorescent nuclei correspond primarily to non-dividing asymmetric daughter cells produced during the first 24 hours of p53-induction, as well as an indeterminate fraction produced early in the BrdU incorporation period.

In Silico Analyses

The NCI60 database was downloaded from http://genome-www.stanford.edu/cgi-bin/sutech/data/download/nci60/index.html. The normalized Cy5/Cy3 ratio parameter “RAT2N” was used for all analyses (Ross et al. 2000).

All statistical analyses were performed using the statistical analysis software StatView® (SAS Institute, Inc.).

TABLE 2
Percent Hoechst-Bright Nuclei Fraction as an Indicator of
Asymmetric Cell Kinetics in Cell Lines with Different
Levels of p53 and IMPDH Expression

| | Percent Hoechst-Bright Nuclei¹ | |
|---|---|---|
| Cell Line | Non-induced | p53-Induced |
| Epithelial Lines² | | |
| Non-inducible control, 1g-1 | 0.0 | 5.0 |
| p53-inducible, 1h-3 | 0.0 | 24.0 |
| Fibroblast Lines³ | | |
| p53-null control, Con-3 | 0.0 | 0.9 |
| p53-inducible | | |
| Ind-4 | 0.3 | 43.0 |
| Ind-8 | 0.3 | 46.0 |
| Vector-transfectant derivatives of Ind-8 | | |
| tC-2 | 0.2 | 47.0 |
| tC-4 | 0.0 | 24.0 |
| Impd-transfectant derivatives of Ind-8 | | |
| tI-1 | 0.2 | 3.0 |
| tI-3 | 0.2 | 0.2 |
| tI-5 | 0.2 | 6.3 |

¹The percentage of cells with Hoechst-bright nuclei fraction was determined as described in Methods.
²p53-inducible mammary epithelial cell lines induced by culture at 32.5° C. (Sherley 1991).
³p53-inducible murine embryo fibroblast cell lines induced by culture in medium containing 75 μM ZnCl₂ (Liu et al. 1 998ab).

TABLE 3
Estimate of the Magnitude of the Change in the Probability of
Asymmetric Cell Kinetics (ΔPack) Associated with Changes in
p53 and IMPDH Expression.

| Regression Slope | Δx/Δy | n | R² | p |
|---|---|---|---|---|
| ΔFd/Δ[Zn] | −0.017/μM | 5 | 0.967 | 0.007 |
| Δp53/Δ[Zn9 | 0.006 μM | 8 | 0.667 | 0.013 |
| ΔIMPDH protein/Δ[Zn] | −0.003/μM | 6 | 0.765 | 0.023 |
| ΔIMPDH activity/Δ[Zn] | −0.002/μM | 5 | 0.818 | 0.035 |

| Calculated | ΔPack /Δy |
|---|---|
| (ΔPack/ΔFd = −2.1; see text) | |
| ΔPack/Δp53 = ΔPack/ΔFd × 1/(Δp53/Δ[Zn]) × ΔFd/Δ[Zn] = | 6 |
| ΔPack/ΔIMPDH protein = ΔPack/ΔFd × 1/(ΔIMPDH protein/Δ[Zn]) × ΔFd/Δ[Zn = | −12 |
| ΔPack/ΔIMPDH activity = ΔPack/ΔFd × 1/(ΔIMPDH activity/Δ[Zn]) × ΔFd/ΔZn] = | −18 |
| Mean \|ΔPack/Δy\| = | 12 |

(p = 0.037*)
*One-tail Student's t-test

### REFERENCES

Berardi, A. C., Wang, A., Levine, J. D., Lopez, P., Scadden, D. T. (1995) Functional isolation and characterization of human hematopoetic stem cells. Science 267, 104-108.

Bernstein, A., Dick, J. E., Huszar, D., Robson, I., Rossant, J., Magli, C., Estrov, Z., Freeman, M., and Phillips, R. A. (1986) Genetic engineering of mouse and human stem cells. Cold Spring Harbor Symposia on Quantitative Biology LI, 1083-1091.

Brenner, M. K. (1996). Gene transfer to hematopoetic cells. New Engl. J. Med. 335, 337-339.

Cairns, J. (1975). Mutation selection and the natural history of cancer. Nature 255, 197-200.

Cheshier, S. H., Morrison, S. J., Liao, X. and Weissman, I. L. (1999) In vivo proliferation and cell cycle kinetics of long-term self-renewing hematopoetic stem cells. Proc. Natl. Acad. Sci. USA 96, 3120-3125.

Collart, F. R., Chubb, C. B., Mirkin, B. L., and Huberman, E. (1992) Increased inosine-5′-phosphate dehydrogenase gene expression in solid tumor tissues and tumor cell lines. Cancer Res. 52, 5826-5828.

Dearden, P. and Akam, M. (2000) Segmentation in silico. Nature 406, 131-132.

El-Deiry, W. S., Tokino, T., Velculescu, V. E., Levy, D. B., Parsons, R., Trent, J. M., Lin, D., Mercer, W. E., Kinzler, K. W., and Vogelstein, B. (1993) WAF1, a potential mediator of p53 tumor suppression. Cell 75, 817-825.

Epperly, M. W., Bray, J. A., Carlos, T. M., Prochownik, E., and Greenberger, J. S. (1999) Biology of marrow stromal cell lines derived from long-term bone marrow cultures of Trp53-deficient mice. Radiation Res. 152, 29-40.

Fuchs, E. and Segre, J. A. (2000) Stem cells: A new lease on life. Cell 100, 143-155.

Giaccia, A. J. and Kastan, M. B. (1998) The complexity of p53 modulation: emerging patterns from divergent signals. Genes & Dev. 12, 2973-2983.

Goodell, M. A., Brose, K., Paradis, G., Conner, A. S., and Mulligan, R. C. (1996) “Isolation and functional properties of murine hematopoetic stem cells that are replicating in vivo.” J. Exp. Med. 183, 1797-1806.

Goodell, M. A., Rosenzweig, M., Kim, H., Marks, D. F., DeMaria, M., Paradis, G., Grupp, S. A., Sieff, C. A., Mulligan, R. C. and Johnson, R. P. (1997) Dye efflux studies suggest that hematopoetic stem cells expressin low or undetectable levels of CD34 antigen exist in multiple species. Nature Med. 3, 1337-1345.

Gottleib, T. M. and Oren, M. (1996) p53 in growth control and neoplasia. Biochim. Biophys. Acta 1287, 77-102.

Greenberger, J. S., Epperly, M. W., Zeevi, A., Brunson, K. W., Goltry, K. L., Pogue-Geile, K. L., Bray, J. and Berry, L. (1996) Stromal cell involvement in leukemogenesis and carcinogenesis. in vivo 10, 1-18.

Gridelli, B. and Remuzzi, G. (2000) Strategies for making more organs available for transplantation. New Engl. J. Med. 343, 404-410.

Grisham, J. W. and Thorgeirsson, S. S. (1997) Liver stem cells. In Stem Cells, C. S. Potten, ed. (San Diego, Calif.: Harcourt Brace & Co.), pp.1-28.

Gu, J. J., Stegmann, S., Gathy, K., Murray, R., Laliberte, J., Ayscue, L., and Mitchell, B. S. (2000) Inhibition of T lymphocyte activation in mice heterozygous for loss of the IMPDH gene. J. Clin. Invest. 106, 599-606.

Hauschka, P. V. (1973) “Analysis of nucleotide pools in animal cells” in Methods in Cell Biology, Prescott, D. M., ed. Academic Press (New York) Vol. VII, pp. 361-462.

Herrero-Jimenez, P., Thilly, G., Southam, P. J., Tomita-Mitchell, A., Morgenthaler, S., Furth, E. E., and Thilly, W. G. (1998). Mutation, cell kinetics, and subpopulations at risk for colon cancer in the United States. Mutation Res. 400, 553-578.

Hollstein, M., Sidransky, D., Vogelstein, B., and Harris, C. C. (1991) p53 mutations in human cancers. Science 253, 49-53.

Huang, S., Law, P., Francis, K., Palsson, B. O., and Ho, A. D. (1999) “Symmetry of initial cell divisions among primitive hematopoetic progenitors is independent of ontogenic age and regulatory molecules.” Blood 94, 2595-2604.

Jackson, R. C., Weber, G., and Morris, H. P. (1975) IMP dehydrogenase, an enzyme linked with proliferation and malignancy. Nature 256, 331-333.

Jardim, A., Bergeson, S. E., Shih, S., Carter, N., Lucas, R. W., Merlin, G., Myler, P. J., Stuart, K., and Ullman, B. (1999) Xanthine phosphoribosyltransferase from Leishmani donovani. Molecular cloning, biochemical characterization, and genetic analysis. J. Biol. Chem. 274, 34403-34410.

Jordan, C. T. and Lemischka, I. R. (1990) Clonal and systemic analysis of long-term hematopoiesis in the mouse. Genes Dev. 4, 220-232.

Knudson, A. G. (1992) Stem cell regulation, tissue ontogeny, and oncogenic events. Seminars in Can. Biol. 3, 99-106.

Kornberg, A. (1980) DNA replication, ed. 2. W. H. Freeman and Co., San Francisco, Calif.

Latham, K. M., S. W. Eastman, A. Wong, and P. W. Hinds. (1996). Inhibition of p53-mediated growth arrest by overexpression of cyclin-dependent kinases. Mol. Cell. Biol. 16, 4445-4455.

Latt, S. A., George, Y. S., and Gray, J. W. (1977) Flow cytometric analysis of bromodeoxyuridine-substituted cells stained with 33258 Hoechst J. Histochem. Cytochem. 25, 927-934.

Leong, P.-M., Thilly, W. G., and Morgenthaler, S. (1985) Variance estimation in single-cell mutation assays: comparison to experimental observations in human lymphoblasts at 4 gene loci.

Levine, A. J. and Momand, J. (1990) Tumor suppressor genes: the p53 and retinoblastoma sensitivity genes and gene products. Biochim. Biophys. Acta 1032, 119-136.

Lin, D., Fiscella, M., O'Connor, P. M., Jackman, J., Chen, M., Luo, L. L., Sala, A., Travali, S., Appella, E., Mercer, W. E. (1994). Constitutive expression of B-myb can bypass p53-induced Waf1/Cip1-mediated G1 arrest. Proc. Natl. Acad. Sci. USA 91, 10079-10083.

Liu, X., Constantinescu, S. N., Sun, Y., Bogan, J. S., Hirsch, D., Weinberg, R. A., and Lodish, H. F. (2000) Generation of mammalian cells stably expressing murine genes at predetermined levels. Anal. Biochem. 280, 20-28.

Liu, Y., Bohn, S. A., and Sherley, J. L. (1998a) Inosine-5′-monophosphate dehydrogenase is a rate-determining factor for p53-dependent growth regulation. Mol. Biol. Cell 9, 15-28.

Liu, Y., Riley, L. B., Bohn, S. A., Boice, J. A., Stadler, P. B., and Sherley, J. L. (1998b) Comparison of Bax, Waf1, and IMP dehydrogenase regulation in response to wild-type p53 expression under normal growth conditions. J. Cellular Physiology 177, 364-376.

Loeffler, M. and Potten, C. S. (1997). Stem cells and cellular pedigrees—a conceptual introduction. In Stem Cells, C. S. Potten, ed. (San Diego, Calif.: Harcourt Brace & Co.), pp.1-28.

Matioli, G., Niewisch, H., and Vogel, H. (1970) Stochastic stem cell renewal. Rev. Europ. Etudes Clin. Et Biol. XV, 20-22.

Maurer, S. M., Firestone, R. B., and Scriver, C. R. (2000) Science's neglected legacy. Nature 405, 117-120.

Merok, J. L. and Sherley, J. L. (2001) Breaching the Kinetic Barrier to In Vitro Somatic Stem Cell Propagation. J. Biomed. Biotech 1, 24-26.

Mitaka, T., Sato, F., Mizuguchi, T., Yokono, T., Mochizuki, Y. (1999) Reconstruction of hepatic organoid by rat small hepatocytes and hepatic nonparenchymal cells. Hepatology 29, 111-125.

Moore, K. A., Ema, H., and Lemischka, I. R. (1997) In vitro maintenance of highly purified, transplantable hematopoetic stem cells. Blood 89, 4337-4347.

Murray, A. W. (1971) The biological significance of purine salvage. Ann. Rev. Biochem. 40, 811-826.

Nagai, M., Natsumeda, Y., and Weber, G. (1992) Proliferation-linked regulation of type II IMP dehydrogenase gene in human normal lymphocytes and HL-60 leukemic cells. Cancer Res. 52, 258-261.

Natsumeda, Y., Ohno, S., Kawasaki, H., Konno, Y., Weber, G., and Suzuki, K. (1990) Two distinct cDNAs for human IMP dehydrogenase. J. Biol. Chem. 265, 5292-5295. McKay, R. (2000) Stem cells-hype and hope. Nature 406, 361-364.

Neutra, M. and Louvard, D. (1989) “Differentiation of intestinal cells in vitro”, In Epithelial Cells in Culture, Alan R. Liss, pp. 363-398.

Nias, A. H. W. and Lajtha, L. G. (1965) “Clone size distribution in the study of inhomogeneity of growth rates in tissue culture” in Cell Culture, C. V. Ramakrishnan, ed. (Dr. W. Junk Publishers, Netherlands).

O'Connor, P. M., Jackman, J., Bae, I., Myers, T. G., Fan, S., Mutoh, M., Scudiero, D. A., Monks, A., Sausville, E. A., Weinstein, J. N., Friend, S., Formace, Jr., A. J., and Kohn, K. W. (1997) Characterization of the p53 tumor suppressor pathway in cell lines of the National Cancer Institute anticancer drug screen and correlations with the growth-inhibitory potency of 123 anticancer agents. Cancer Res. 57, 4285-4300.

Petersen, B. E., Bowen, W. C., Patrene, K. D., Mars, W. M., Sullivan, A. K., Murase, N., Boggs, S. S., Greenberger, J. S., Goff, J. P. (1999) Bone marrow as a potential source of hepatic oval cells. Science 284, 1168-1170.

Phillips, R. L., Ernst, R. E., Brunk, B., Ivanova, N., Mahan, M. A., Deanehan, J. K., Moore, K. A., Overton, G. C., and Lemischka, I. R. (2000) The genetic program of hematopoetic stem cells. Science 288, 1635-1640.

Poldosky, D. K. (1993). Regulation of intestinal epithelial proliferation: a few answers, many questions. Am. J. Physiol. 264, G179-G186.

Potten, C. S. and Grant, H. K. (1998). The relationship between ionizing radiation-induced apoptosis and stem cells in the small and large intestine. British J. of Cancer 78, 993-1003.

Potten, C. S. and Morris, R. J. (1988) Epithelial stem cells in vivo. J. Cell Sci. Suppl. 10, 45-62.

Powers, M. J., Rodriguez, R. E., Griffith, L. G. (1997) Cell-substratum adhesion strength as a determinant of hepatocyte aggregate morphology. Biotech. and Bioeng. 53, 415-426.

Puck, T. T. and Marcus, P. I., J. (1956) Experimental Medicine 103, 653.

Rambhatla, L., Bohn, S. A., Stadler, P. B., Boyd, J. T., Coss, R. A., and Sherley, J. L. (2000). Cellular senescence: ex vivo p53-dependent asymmetric cell kinetics. J. Biomed. Biotech, 1, 27-36.

Reisner, Y., Itzicovitch, L., Meshorere, A., and Sharon, N. (1978). Hematopoetic stem cell transplantation using mouse bone marrow and spleen cells fractionated by lectins. Proc. Natl. Acad. Sci. USA 75, 2933-2936.

Ross D. T., Scherf, U., Eisen, M. B., Perou, C. M., Rees, C., Spellman, P., Iyer, V., Jeffrey, S. S., Van de Rijn, M., Waltham, M., Pergamenschikov, A., Lee, J. C., Lashkari, D., Shalon, D., Myers, T. G., Weinstein, J. N., Botstein, D., and Brown, P. O. (2000) Systematic variation in gene expression patterns in human cancer cell lines. Nat. Genet. 24, 227-235.

Senda, M. and Natsumeda, Y. (1994) Tissue-differential expression of two distinct genes for human IMP dehydrogenase (E.C.1.1.1.205). Life Sci. 54, 1917-1926.

Sherley, J. L. (1991) Guanine nucleotide biosynthesis is regulated by the cellular p53 concentration. J. Biol. Chem. 266, 24815-24828.

Sherley, J. L., Stadler, P. B. and Johnson, D. R. (1995a) “Expression Of The Wildtype p53 Antioncogene Induces Guanine Nucleotide-Dependent Stem Cell Division Kinetics.” Proc. Natl. Acad. Sci. USA 92, 136-140.

Sherley, J. L., Stadler, P. B. and Stadler, J. S. (1995b) “A Quantitative Method for the Analysis of Mammalian Cell Proliferation in Culture in Terms of Dividing and Non-dividing cells.” Cell Proliferation 28, 137-144.

Sherley, J. L. (1996) The p53 tumor suppressor gene as regulator of somatic stem cell renewal division. Cope 12, 9-10.

Sherley, J. L. (2000). Tumor Suppressor Genes and Cell Kinetics in the Etiology of Malignant Mesothelioma” in Sourcebook of Asbestos Diseases, G. A. Peters & B. J. Peters, eds. Peters and Peters (Santa Monica), Vol. 21, pp. 91-141.

Sherley, J. L., Stadler, P. B., and D. R. Johnson (1995a). Expression of the wildtype p53 antioncogene induces guanine nucleotide-dependent stem cell division kinetics. Proc. Natl. Acad. Sci. 92, 136-140.

Sherley, J. L., Stadler, P. B., and Stadler, J. S. (1995b) A quantitative method for the analysis of mammalian cell proliferation in culture in terms of dividing and non-dividing cells. Cell Prolif. 28, 137-144.

Smaglik, P. (2000) “Embryo stem-cell work gets NIH go-ahead”, In Nature 406, p. 925.

Stadler, P. B., Pennacchi, J., and Sherley, J. L. (1994) Inosine-5′-monophosphate dehydrogenase activity is maintained in immortalized murine cells growth-arrested by serum deprivation. Adv. Enzyme Regul. 34, 91-106.

Tiedeman, A. A. and Smith, J. M. (1991) Isolation and sequence of a cDNA encoding mouse IMP dehydrogenase. Gene 97, 289-293.

Weissman, I. L. (2000) Stem cells: units of development, units of regeneration, and units of evolution. Cell 100, 157-168.

Wilson, J. M. (1993). Vehicles for gene therapy. Nature 365, 691-692.

All references described herein are incorporated herein by reference.

I claim:

1. A method of culturing and expanding somatic stem cells in vitro, comprising culturing somatic stem cells isolated from a mammal in a culture medium which permits cell growth under conditions and for a time sufficient to permit cell growth, wherein a guanine nucleotide biosynthesis pathway in said somatic stem cells is enhanced by an agent present in the culture medium or by a genetic manipulation to said somatic stem cells.

2. The method of claim 1, wherein an agent is present in said media.

3. The method of claim 2, wherein said agent is a guanine nucleotide precursor, an analogue or derivative thereof.

4. The method of claim 3, wherein said guanine nucleotide precursor is xanthosine or hypoxanthine.

5. The method of claim 2, wherein said agent is xanthine.

6. The method of claim 3, wherein said guanine nucleotide precursor is xanthosine.

7. The method of claim 3, wherein said guanine nucleotide precursor is present in an amount of 1-5000 μM.

8. The method of claim 7, wherein said guanine nucleotide precursor is present in an amount of 50-400 μM.

9. The method of claim 1, wherein genetic manipulation is used.

10. The method of claim 9, wherein the genetic manipulation results in upregulation of guanine nucleotide biosynthesis.

11. The method of claim 10, wherein the genetic manipulation comprises expressing a gene encode inosine-5′monophosphate dehydrogenase (IMPDH) or xanthine phosphoribosyltransferase (XPRT) in the cultured somatic stem cells.

12. The method of claim 11, wherein the gene encodes xanthine phosphoribosyltransferase.

13. The method of claim 1, wherein the somatic stem cell is selected from the group consisting of bone marrow derived stem cells, adipose derived stem cells, mesenchymal stem cells, neural stem cells, liver stem cells, pancreatic stem cells, skin stem cells, and corneal epithelim stem cells.

14. The method of claim 1, wherein cells are cultured at a high cell density.

15. A method for administering somatic stem cells to a subject, wherein said method comprises:
(a) isolating somatic stem cells from said individual or a matched individual;
(b) culturing said isolated somatic stem cells in a medium and under conditions sufficient for culturing;
(c) adding a substituent to said medium to enhance guanine nucleotide biosynthesis suppressing asymmetric kinetics;
(d) culturing said isolated somatic stem for at least 10 days after said substituent is added to expand said isolated somatic cells; and,
(e) administering said isolated stem cells of step (d) to said individual.

16. A method for deriving clonal cells lines of somatic stem cells by isolating somatic stem cells from a mammal, performing limiting dilution plating or cell sorting of said somatic stem cells to isolate single somatic stem cells, and culturing and expanding said single somatic stem cells using the method of claim 1.

17. A method for identifying molecular probes specific for somatic stem cells, comprising culturing and expanding said single somatic stem cells using the method of claim 1, and using said population of expanded somatic stem cells for comparison to a second population of non-stem cells to identify differences in gene and/or protein expression between the two said populations.

18. A method of culturing and expanding somatic stem cells in vitro, comprising culturing somatic stem cells isolated from a mammal in a culture medium which permits cell growth under conditions and for a time sufficient to permit cell growth, wherein the expression of a protein downstream of the guanine nucleotide biosynthesis pathway in said somatic stem cells is modulated by an agent present in the culture medium or by a genetic manipulation to said somatic stem cells such that asymmetric cell kinetics are suppressed.

19. The method of claim 18, wherein the modulation is increased expression of the protein.

20. The method of claim 18, wherein the modulation is decreased expression of the protein.

21. A method of promoting wound repair in a patient in need thereof, comprising inducing exponential cell kinetics in tissue stem cells by administering to said patient an agent that enhances the guanine nucleotide biosynthesis pathway.

22. The method of claim 21, wherein said agent is a guanine nucleotide precursor or an analogue or derivative thereof.

23. The method of claim 21, wherein said guanine nucleotide precursor is xanthosine or hypoxanthine or an analogue or derivative thereof.

24. The method of claim 23, wherein said agent is xanthine.
